(19) United States
(12) Patent Application Publication
(10) Pub. No.: US 2020/0261440 A1
(43) Pub. Date: Aug. 20, 2020

(54) ZINC IONOPHORES AND USES THEREOF

(71) Applicants: THE UNIVERSITY OF QUEENSLAND, St Lucia, Queensland (AU); THE UNIVERSITY OF ADELAIDE, South Australia (AU); GRIFFITH UNIVERSITY, Nathan, Queensland (AU)

(72) Inventors: Mark WALKER, Toowong, Queensland (AU); Christopher MCDEVITT, Melbourne, Victoria (AU); Mark VON ITZSTEIN, Gilston, Queensland (AU); Alastair MCEWAN, Fig Tree Pocket, Queensland (AU)

(21) Appl. No.: 16/755,328

(22) PCT Filed: Oct. 12, 2018
(86) PCT No.: PCT/AU2018/051116

§ 371 Date: Apr. 10, 2020

(30) Foreign Application Priority Data
Oct. 13, 2017 (AU) 2017904135

(51) Int. Cl. A61K 31/473; A61K 45/06; A61K 38/12; A61K 33/30; A61P 31/04

(52) U.S. Cl. CPC A61K 31/473; A61K 45/06; A61P 31/04; A61K 33/30; A61K 38/12

(57) ABSTRACT

This invention relates to the use of zinc(II) salts in combination with a zinc ionophore to resensitize a previously resistant pathogenic bacteria to an antibiotic. Methods of restoring the sensitivity of a resistant pathogenic bacterium to an antibiotic comprising administering a zinc ionophore in combination with a zinc(II) salt and methods of treating a bacterial infection comprising administering a zinc ionophore in combination with a zinc (II) salt concurrently and/or sequentially with administration of a therapeutically effective amount of an antibiotic is also described.

This application claims priority to Australian Provisional Application No. 2017904135 entitled “Compositions and their uses” filed 13 Oct. 2017, the contents of which are incorporated herein by reference in their entirety.

### BACKGROUND OF THE INVENTION

The present invention relates to use of zinc ionophores, or zinc(II) salts in combination with zinc ionophores, as antibiotic adjuvants or potentiators. Their use in restoring the sensitivity of one or more resistant bacteria to one or more antibiotics is also described. Pharmaceutical compositions comprising an antibiotic in combination with a zinc ionophore, a zinc(II) coordination complex, or a zinc(II) salt in combination with a zinc(II) ionophore, together with methods of treating bacterial infections are also described.

### DESCRIPTION OF THE PRIOR ART

The range of antibiotics available to combat bacterial infections is diminishing due to the development of bacterial resistance to all classes of antibiotics. The number of new classes of antibiotics is decreasing, and the pharmaceutical pipeline is diminishing [see, e.g. Cooper, M. A. & Shlaes, D. Fix the antibiotics pipeline. Nature 472, 32, doi:10.1038/472032a (2011); Chakradhar, S. What's old is new: Reconfiguring known antibiotics to fight drug resistance. Nat Med 22, 1197-1199, doi:10.1038/nm1116-1197 (2016)].

There is an emergence of resistant bacteria on a global scale, and the World Health Organisation reports that antibiotic resistant pathogens represent an imminent global health threat (World Health Organization. Antimicrobial resistance: global report on surveillance 2014., http://www.who.int/drugresistance/documents/surveillancereport/en/). The United States Center for Disease Control and Prevention categorises erythromycin-resistant Group A Streptococcus spp. (GAS), methicillin-resistant Staphylococcus aureus (MRSA), and vancomycin-resistant Enterococcus (VRE), as concerning or serious threats to human health.

Antibiotic resistance is the ability of bacteria to resist antibiotic medication used to treat bacterial infections. Antibiotic resistance occurs when bacteria are intrinsically resistant to an antibiotic or when bacteria change in response to use of antibiotics allowing them to inactivate the antibiotic, resist the action of the antibiotic, or remove or exclude the antibiotic from the bacterial cell. An increasing number of antibacterial infections such as pneumonia, tuberculosis and gonorrhoea are becoming more difficult to treat as the antibiotics previously used successfully to treat them become less effective due to antibiotic resistance. Antibiotic resistance results in longer hospital stays, higher medical costs and increased mortality.

The mechanism through which pathogenic bacteria develop resistance depends on several factors which can include the class and structure of antibiotic, and the nature of the pathogenic bacteria.

As the level and frequency of bacterial resistance to last generation antibiotics increases, the need to develop alternative therapies has become more urgent. Strategies that are being explored to address the problem of bacterial resistance to existing antibiotics include the development of new antibiotics, blocking of resistance mechanisms against existing antibiotics, bacteriophage therapy, targeting of virulence mechanisms that weaken bacterial defence against host immunity, stimulating host immunity to improve bacterial clearance, destabilization of the Gram-negative cell envelope and repurposing existing drugs used for non-infectious disease indications [see, e.g. Ling, L. L. et al. A new antibiotic kills pathogens without detectable resistance. Nature 520, 388, doi:10.1038/nature14303 (2015); Vaara, M. & Vaara, T. Sensitization of Gram-negative bacteria to antibiotics and complement by a nontoxic oligopeptide. Nature 303, 526-528 (1983); Fischbach, M. A. & Walsh, C. T. Antibiotics for emerging pathogens. Science 325, 1089-1093, doi:10.1126/science.1176667 (2009); Summers, W. C. Bacteriophage therapy. Annu Rev Microbiol 55, 437-451, doi:10.1146/annurev.micro.0.55.1.437 (2001); Zinkernagel, A. S., Peyssonnaux, C., Johnson, R. S. & Nizet, V. Pharmacologic augmentation of hypoxia-inducible factor-1alpha with mimosine boosts the bactericidal capacity of phagocytes. J Infect Dis 197, 214-217, doi:10.1086/524843 (2008); and Gill, E. E., Franco, 0. L. & Hancock, R. E. Antibiotic adjuvants: diverse strategies for controlling drug-resistant pathogens. Chem Biol Drug Des 85, 56-78, doi:10.1111/cbdd.12478 (2015)].

Bacterial pathogens such as GAS, MRSA and VRE are responsible for a wide range of hospital-acquired and community-acquired infections. These infections place significant pressure and economic burden even on established healthcare systems, and are a major contributor to global human morbidity and mortality [see, Woolhouse, M., Waugh, C., Perry, M. R. & Nair, H. Global disease burden due to antibiotic resistance—state of the evidence. J Glob Health 6, 010306, doi:10.7189/jogh.06.010306 (2016)].

There is a need to identify further viable clinically useful adjuvants to restore the sensitivity of resistant bacteria to antibiotics.

### SUMMARY OF THE PRESENT INVENTION

The present invention is predicated at least in part on the surprising discovery that certain zinc ionophores, or certain combinations of a zinc(II) salt and a zinc ionophore, have the ability to restore the sensitivity of one or more antibiotic resistant pathogenic bacteria to one or more antibiotics.

Accordingly, in one aspect the present invention advantageously provides the use of a zinc ionophore in combination with a pharmaceutically acceptable zinc(II) salt or solvate thereof for restoring the sensitivity of a resistant pathogenic bacterium to an antibiotic. In certain embodiments, the zinc ionophore is pharmaceutically acceptable. In some embodiments, the zinc ionophore and zinc(II) salt are used in combination with an antibiotic. In certain embodiments, the antibiotic is not an aminoglycoside antibiotic.

In another aspect, the present invention provides the use of a zinc ionophore in combination with a pharmaceutically acceptable zinc(II) salt or solvate thereof for inhibiting resistance of a pathogenic bacterium to an antibiotic. Preferably the antibiotic is not an aminoglycoside antibiotic. The present invention also provides the use of a zinc ionophore in combination with a pharmaceutically acceptable zinc(II) salt or solvate thereof, as an antibiotic adjuvant or antibiotic potentiator. In some embodiments, the antibiotic is not an aminoglycoside antibiotic. In some embodiments the zinc ionophore and zinc(II) salt are used in combination with an antibiotic.

In yet another aspect, the invention also provides a method of restoring the sensitivity of a resistant pathogenic bacterium to an antibiotic comprising administering an effective amount of a zinc ionophore in combination with an effective amount of a pharmaceutically acceptable zinc(II) salt to a subject in need thereof. In some embodiments the method also includes administering an antibiotic.

In some embodiments the zinc ionophore and zinc(II) salt are used in combination with an antibiotic. In some embodiments, the antibiotic is not an aminoglycoside antibiotic. In some embodiments the zinc ionophore is pharmaceutically acceptable. In some embodiments the zinc ionophore is in the form of a pharmaceutically acceptable derivative.

In a yet further aspect, the present invention provides a pharmaceutical formulation comprising a pharmaceutically acceptable zinc ionophore and a pharmaceutically acceptable zinc(II) salt or a solvate thereof; and, optionally, an antibiotic or a pharmaceutically acceptable derivative thereof;

and a pharmaceutically acceptable carrier.
In some embodiments, the antibiotic is not an aminoglycoside antibiotic. In some embodiments, the pharmaceutical composition is for restoring the sensitivity of a resistant pathogenic bacterium to an antibiotic. In some embodiments, the pharmaceutical formulation is for inhibiting resistance of a pathogenic bacterium to an antibiotic. In some embodiments, the pharmaceutical composition is for use in treating a bacterial infection.

The present invention also provides a use of a pharmaceutically acceptable zinc ionophore and a pharmaceutically acceptable zinc(II) salt or solvate thereof, or a zinc(II) coordination complex or a pharmaceutically acceptable derivative thereof, in the manufacture of a medicament for resensitizing a resistant pathogenic bacterium to an antibiotic, or for inhibiting resistance of a pathogenic bacterium to an antibiotic. There is further provided a use of a pharmaceutically acceptable zinc ionophore and a pharmaceutically acceptable zinc(II) salt or solvate thereof, or a zinc(II) coordination complex or a pharmaceutically acceptable derivative thereof, in the manufacture of a medicament for separate, sequential or simultaneous use with an antibiotic or a pharmaceutically acceptable derivative thereof for treatment of a bacterial infection. There is also provided a use of a pharmaceutically acceptable zinc ionophore and a pharmaceutically acceptable zinc(II) salt or solvate thereof, or a zinc(II) coordination complex or a pharmaceutically acceptable derivative thereof, in combination with an antibiotic or pharmaceutically acceptable derivative thereof, in the manufacture of a medicament for treatment of a bacterial infection.

In another aspect, there is further provided a pharmaceutically acceptable zinc ionophore and a pharmaceutically acceptable zinc(II) salt or solvate thereof, or a zinc(II) coordination complex or a pharmaceutically acceptable derivative thereof, for use in restoring the sensitivity of a resistant pathogenic bacterium to an antibiotic, or for use in inhibiting resistance of a pathogenic bacterium to an antibiotic. There is also provided a pharmaceutically acceptable zinc ionophore and a pharmaceutically acceptable zinc(II) salt or solvate thereof, or a zinc(II) coordination complex or a pharmaceutically acceptable derivative thereof, for separate, sequential or simultaneous use with an antibiotic for treatment of a bacterial infection. There is also provided a pharmaceutically acceptable zinc ionophore and a pharmaceutically acceptable zinc(II) salt or solvate thereof, or a zinc(II) coordination complex or a pharmaceutically acceptable derivative thereof, in combination with an antibiotic or a pharmaceutically acceptable derivative thereof for treatment of a bacterial infection.

In yet another aspect, the present invention provides a pharmaceutical formulation comprising a pharmaceutically acceptable zinc ionophore and a pharmaceutically acceptable zinc(II) salt or solvate thereof; and a pharmaceutically acceptable carrier; for use in restoring the sensitivity of a resistant pathogenic bacterium to an antibiotic, or for use in inhibiting resistance of a pathogenic bacterium to an antibiotic; wherein the antibiotic is not an aminoglycoside antibiotic.

It has now been discovered that, in at least one embodiment, a zinc(II) salt in combination with a zinc ionophore, or a zinc(II) coordination complex, in accordance with the invention can restore the sensitivity of one or more species of pathogenic bacteria to one or more antibiotics, or can inhibit resistance of one or more pathogenic bacteria to one or more antibiotics. Accordingly, certain combinations of zinc(II) salts and zinc ionophores, optionally in the form of a zinc coordination complex, can be used in combination with certain antibiotics to treat bacterial infections caused by one or more pathogenic bacteria which has previously developed resistance to that antibiotic. In some embodiments, the molar ratio of zinc(II) salt to zinc ionophore is approximately 1:2, or approximately 1:1. In some embodiments, the combination of zinc(II) salt and zinc ionophore comprises an excess of zinc(II) salt, for example a stoichiometric excess of zinc(II) salt.

Without being bound by the theory, it is believed that the zinc ionophore or the ligands of the zinc(II) co-ordination complex can “mask” the electronic charge on the zinc(II) cation to allow the zinc ion to diffuse across a lipophilic bacterial cell membrane more easily. After the zinc(II) ion/ionophore has been transported into the bacterial cell, the combination is believed to exhibit antibacterial effects by destabilizing metal homeostasis. In some embodiments, in addition to changes in the transcription of heavy metal homeostasis genes, the transcription of several essential virulence and metabolic systems was also observed to be disrupted by sub-inhibitory concentrations of zinc(II) ion/ionophore. In some embodiments, these disruptions are believed to enhance antibiotic sensitivity in otherwise resistant bacterial pathogens.

It has also been discovered that one or more zinc ionophores according to the invention have the ability to restore the sensitivity of one or more antibiotic resistant pathogenic bacteria to one or more antibiotics, or inhibit resistance of one or more pathogenic bacteria to one or more antibiotics, in the absence of a zinc(II) salt.

Accordingly, in some embodiments, there is provided compositions, methods and uses according to the invention wherein the zinc ionophore is used in the absence of a zinc(II) salt. In some embodiments, the zinc ionophore is the sole antibiotic adjuvant or antibiotic potentiator present. In some embodiments, the zinc ionophore is used in the absence of additional zinc(II) salt. In some embodiments, the zinc(II) salt is used in the absence of another antibiotic adjuvant or antibiotic potentiator.

In some aspects, the present invention also provides a use of a zinc ionophore for restoring the sensitivity of a resistant pathogenic bacterium to an antibiotic. In another aspect, there is provided a use of a zinc ionophore for inhibiting resistance of a pathogenic bacterium to an antibiotic. In some embodiments, the zinc ionophore is used in the absence of zinc(II) salt or zinc(II) ions. The present invention also provides the use of a zinc ionophore as an antibiotic adjuvant or antibiotic potentiator. In certain embodiments, the zinc ionophore is pharmaceutically acceptable. In some embodiments the zinc ionophore is used in combination with an antibiotic. In some embodiments, the antibiotic is not an aminoglycoside antibiotic.

In yet another aspect, the present invention also provides a method of restoring the sensitivity of a resistant pathogenic bacterium to an antibiotic, or inhibiting resistance of a pathogenic bacterium to an antibiotic, comprising administering an effective amount of a zinc ionophore to a subject in need thereof. In some embodiments, the zinc ionophore is administered in the absence of an additional source of zinc(II) ions. In some embodiments the method also includes administering an antibiotic.

Inhibition, or restoration, of antibiotic resistance is useful in the treatment of bacterial infection in a subject, for example bacterial infection caused by resistant bacteria. Accordingly, in at least one embodiment, a zinc ionophore; a combination of a zinc(II) salt or a solvate thereof and a zinc ionophore; or a zinc(II) coordination complex according to the present invention are considered useful when administered in combination with an antibiotic for the treatment of bacterial infection.

Accordingly, in another aspect, the present invention also provides a method of treating a bacterial infection in a subject comprising administering to a subject in need thereof an effective amount of a pharmaceutically acceptable zinc ionophore, or a pharmaceutically acceptable zinc ionophore in combination with an effective amount of a pharmaceutically acceptable zinc(II) salt or solvate thereof, or a pharmaceutically acceptable zinc(II) coordination complex, concurrently and/or sequentially with administration of a therapeutically effective amount of an antibiotic or pharmaceutically acceptable derivative thereof. In some embodiments the antibiotic is not an aminoglycoside antibiotic.

There is further provided a method of treating a bacterial infection in a subject comprising the administration of a therapeutically effective and non-toxic amount of a pharmaceutical composition according to the invention.

In a further aspect, the present invention provides the use of a pharmaceutically acceptable zinc ionophore, or a pharmaceutically acceptable zinc ionophore in combination with a pharmaceutically acceptable zinc(II) salt or solvate thereof; or a pharmaceutically acceptable zinc(II) coordination complex, in the manufacture of a medicament for treating a bacterial infection, wherein the medicament is for co-administration together, simultaneously, successively or in any order with an antibiotic or a pharmaceutically acceptable derivative thereof, wherein the antibiotic is not an aminoglycoside antibiotic.

In a yet further aspect, the present invention provides a pharmaceutically acceptable zinc ionophore, or a pharmaceutically acceptable zinc ionophore in combination with a pharmaceutically acceptable zinc(II) salt or solvate thereof; or a pharmaceutically acceptable zinc(II) coordination complex, for use in treating bacterial infection, wherein the use is in combination with an antibiotic. In some embodiments, the use is for co-administration together, simultaneously, successively or in any order with an antibiotic or a pharmaceutically acceptable derivative thereof. In some embodiments, the antibiotic is not an aminoglycoside antibiotic.

In another aspect, the present invention further provides a pharmaceutical composition comprising a pharmaceutically acceptable zinc ionophore, a pharmaceutically acceptable zinc(II) salt or solvate thereof and a pharmaceutically acceptable zinc ionophore, or a pharmaceutically acceptable zinc(II) coordination complex; an antibiotic or a pharmaceutically acceptable derivative thereof; and a pharmaceutically acceptable carrier. In some embodiments the composition is for treatment of a bacterial infection. In a further aspect, the present invention provides a method of treating a bacterial infection in a subject comprising the administration of a therapeutically effective and non-toxic amount of a pharmaceutical composition according to the invention.

There is further provided a pharmaceutical composition comprising a pharmaceutically acceptable zinc ionophore; a pharmaceutically acceptable zinc(II) salt or solvate thereof and a pharmaceutically acceptable zinc ionophore, or a pharmaceutically acceptable zinc(II) coordination complex, for use as an active therapeutic substance in the treatment of a bacterial infection in a subject. In some embodiments, the composition further comprises one or more therapeutically active substances. In some embodiments, a further therapeutically active substance is an antibiotic or a pharmaceutically acceptable derivative thereof. In some embodiments, a further therapeutically active substance is another antibiotic adjuvant or antibiotic potentiator.

In another aspect there is provided a pharmaceutical composition comprising a pharmaceutically acceptable zinc ionophore or a pharmaceutically acceptable derivative thereof, a zinc(II) salt or pharmaceutically acceptable solvate thereof and a pharmaceutically acceptable zinc ionophore or a pharmaceutically acceptable derivative thereof, or a zinc(II) coordination complex or a pharmaceutically acceptable derivative thereof; an antibiotic or a pharmaceutically acceptable derivative thereof; and a pharmaceutically acceptable excipient.

The invention further provides the use of a pharmaceutical composition according to the invention for the treatment of bacterial infection in a subject. There is further provided a pharmaceutical composition according to the invention for use in treating bacterial infection in a subject. The invention also provides the use of a pharmaceutical composition according to the invention as an antibacterial agent. There is further provided a pharmaceutical composition according to the invention for use as an antibacterial agent.

The invention also provides the use of a pharmaceutically acceptable zinc ionophore, a pharmaceutically acceptable zinc(II) salt or solvate thereof and a pharmaceutically acceptable zinc ionophore, or a pharmaceutically acceptable zinc(II) coordination complex, in the manufacture of a medicament for treating a bacterial infection wherein the medicament is for co-administration together, simultaneously, separately, successively or in any order with an antibiotic.

The invention further provides a kit or commercial package comprising, as active components, a combination of a pharmaceutical composition comprising a pharmaceutically acceptable zinc ionophore, or a pharmaceutically acceptable zinc(II) salt or solvate thereof and a pharmaceutically acceptable zinc ionophore, or, alternatively, a pharmaceutically acceptable zinc(II) coordination complex or derivative thereof; and a pharmaceutical composition comprising an antibiotic or a pharmaceutically acceptable derivative thereof, together with instructions for simultaneous, separate or sequential administration of said combination to a patient in need thereof for use in the treatment of bacterial infection.

In some embodiments, the zinc ionophore is an 8-hydroxyquinoline compound, for example an 8-hydroxyquinoline compound as described in WO 2004/007461 which is incorporated by reference herein its entirety. In some embodiments the zinc ionophore is a compound as described in WO 2007/147247 which is incorporated by reference herein in its entirety.

In some embodiments the zinc ionophore is a compound Formula I:

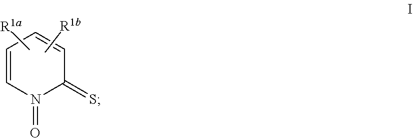

wherein R^{1a} and R^{1b} are independently H, halogen, OR^{2a}, SR^{2a}, CF₃, C₁₋₄alkyl, or NR^{2a}R^{2b};
R^{2a} and R^{2b} are independently H, or optionally substituted C₁₋₄alkyl;
or a pharmaceutically acceptable derivative thereof;
or a compound of Formula II:

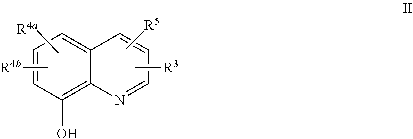

wherein:
R³ and R⁵ are independently H; optionally substituted C₁₋₆ alkyl; optionally substituted C₂₋₆ alkenyl; optionally substituted C₂₋₆alkynyl; optionally substituted C₃₋₆cycloalkyl; optionally substituted aryl; optionally substituted heterocyclyl; CN; OR⁶, SR⁶, COR⁶, CSR⁶, HCNOR⁶ or HCNNR⁶ in which R⁶ is H, optionally substituted C₁₋₆ alkyl, optionally substituted C₂₋₆alkenyl, optionally substituted C₂₋₆alkynyl, optionally substituted C₃₋₆cycloalkyl, optionally substituted aryl or optionally substituted heterocyclyl; NR⁸R⁹ or SO₂NR⁸R⁹ in which R⁸ and R⁹ are independently selected from H, optionally substituted C₁₋₆ alkyl, optionally substituted C₂₋₆alkenyl, optionally substituted C₂₋₆alkynyl, optionally substituted C₃₋₆cycloalkyl, optionally substituted aryl and optionally substituted heterocyclyl; CONR⁹R¹⁰ in which R⁹ is as defined above and R¹⁰ is optionally substituted C₁₋₆ alkyl, optionally substituted C₂₋₆alkenyl, optionally substituted C₂₋₆alkynyl, optionally substituted C₃₋₆cycloalkyl, optionally substituted aryl or optionally substituted heterocyclyl; CH₂CONR⁸R⁹ in which R⁸ and R⁹ are as defined above; and (CH₂)ₙNR⁹R¹¹ in which R⁹ is as defined above and R¹¹ is selected from optionally substituted C₁₋₆ alkyl, optionally substituted C₂₋₆alkenyl, optionally substituted alkynyl, optionally substituted C₃₋₆cycloalkyl, optionally substituted aryl, optionally substituted heterocyclyl and SO₂R¹² in which R¹² is optionally substituted C₁₋₆alkyl, optionally substituted C₂₋₆alkenyl, optionally substituted C₂₋₆alkynyl, optionally substituted C₃₋₆ cycloalkyl, optionally substituted aryl or optionally substituted heterocyclyl, and n is 1 to 6;
R^{4a} and R^{4b} are independently H, optionally substituted C₁₋₄alkyl or halogen; or a pharmaceutically acceptable derivative thereof.

In some embodiments, the zinc ionophore is a compound of Formula III:

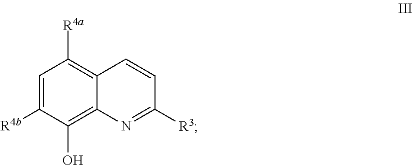

wherein:
R³ is H, optionally substituted C₁₋₆alkyl, CONH₂ or (CH₂)ₙNR⁹R¹¹, wherein n is 0, 1, 2, or 3;
R^{4a} and R^{4b} are independently H, optionally substituted C₁₋₄alkyl or halogen;
R⁹ is H, optionally substituted C₁₋₆ alkyl, optionally substituted C₂₋₆alkenyl, optionally substituted C₂₋₆alkynyl, optionally substituted C₃₋₆cycloalkyl, optionally substituted aryl or optionally substituted heterocyclyl; and
R¹¹ is optionally substituted C₁₋₆ alkyl, optionally substituted C₂₋₆alkenyl, optionally substituted alkynyl, optionally substituted C₃₋₆cycloalkyl, optionally substituted aryl, optionally substituted heterocyclyl or SO₂R¹² in which R¹² is optionally substituted C₁₋₆alkyl, optionally substituted C₂₋₆alkenyl, optionally substituted C₂₋₆alkynyl, optionally substituted C₃₋₆ cycloalkyl, optionally substituted aryl or optionally substituted heterocyclyl; or a pharmaceutically acceptable derivative thereof.

In some embodiments, the zinc ionophore is 5-chloro-7-iodo-8-quinolinol [clioquinol, CQ]:

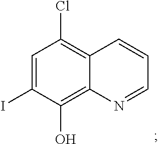 [SMILES: Oc1c(I)cc(Cl)c2cccnc12]

or 5,7-dichloro-2-[(dimethylamino)methyl]-8-quinolinol [PBT2]:

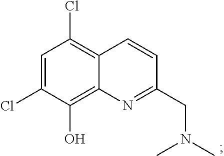 [SMILES: CN(C)Cc1ccc2c(Cl)cc(Cl)c(O)c2n1]

or a pharmaceutically acceptable derivative of either thereof.

In some embodiments, the zinc ionophore and the zinc(II) salt are in the form of a zinc(II) coordination complex, preferably a pharmaceutically acceptable zinc(II) coordination complex, or a pharmaceutically acceptable derivative thereof. In some embodiments, the zinc(II) coordination complex is a compound of Formula VII:

Zn(II)[L]₂ VII

wherein each L is the same and is an anion of a zinc ionophore as defined herein; or a pharmaceutically acceptable derivative thereof.

In some embodiments, the compound of Formula VII is:

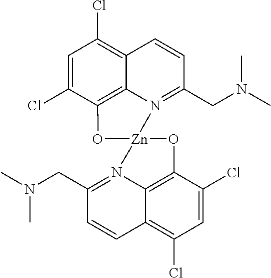

or a pharmaceutically acceptable derivative thereof.

In some embodiments, the antibiotic is a tetracycline, for example tetracycline, doxycycline or tigecycline; or a polypeptide antibiotic, for example a polymyxin, e.g. colistin (polymyxin E) or polymyxin B. In some embodiments the antibiotic is a polypeptide antibiotic, e.g. colistin (polymyxin E) or polymyxin B.

In some embodiments, the zinc(II) salt is ZnCl₂, Zn(CH₃CO₂)₂ or ZnSO₄, and the zinc ionophore is an 8-hydroxyquinoline compound as defined herein, such as clioquinol [CQ] or PBT2. Alternatively the zinc(II) salt and zinc ionophore form a zinc(II) coordination complex Zn(II)[L]₂, wherein each L is the same and is an anion of an ionophore as defined herein. In certain embodiments the antibiotic is a polypeptide antibiotic such as colistin (polymyxin E) or polymyxin B. In some embodiments the pathogenic bacteria is a Klebsiella spp., for example Klebsiella pneumoniae; Escherichia coli; an erythromycin-resistant group A Streptococcus (GAS); a methicillin-resistant Staphylococcus aureus (MRSA); or a vancomycin-resistant Enterococcus (VRE).

In some embodiments of the methods, uses and compositions described herein, the zinc ionophore is used in the absence of a zinc(II) salt. In some embodiments the zinc ionophore is an 8-hydroxyquinoline compound as defined herein, such as clioquinol [CQ] or PBT2. In certain embodiments the antibiotic is a polypeptide antibiotic such as colistin (polymyxin E) or polymyxin B. In some embodiments the pathogenic bacteria is a Klebsiella spp., for example Klebsiella pneumoniae; or Escherichia coli such as MCR1-positive Escherichia coli.

### BRIEF DESCRIPTION OF THE DRAWINGS

FIG. 1: Synergistic antimicrobial activity of PBT2 and zinc.

FIG. 1a illustrates growth of GAS, MRSA and VRE on THY agar in the presence or absence of PBT2 (1.5 μM) and zinc(II) ions (400 μM).

FIG. 1b shows time-kill curves of GAS, MRSA and VRE in THY broth with or without PBT2 (1.5 μM for GAS or 6 μM for MRSA and VRE) and ZnSO₄ (300 μM for GAS 15 and 600 μM for MRSA and VRE). Error bars indicate standard deviation from 2 biological replicates.

FIG. 1c is a graphical illustration of the development of resistance during serial passage in the presence of sub-inhibitory concentrations of antimicrobial compounds in CAMHB. Data represents mean of 3 biological replicates.

FIG. 1d shows CFUs recovered from a murine skin infection model 4 days after challenge with GAS, or MRSA. Mice were treated twice daily with ointment only or ointment with 5 mM PBT2 and/or 50 mM ZnSO₄ (MRSA) or ZnCl₂ (GAS). Data is representative of two independent experiments, values for individual mice are plotted (*=P<0.05, unpaired t-test, non parametric).

FIG. 2: PBT2 and zinc affect heavy metal homeostasis, metabolism and virulence.

FIG. 2a shows the RNAseq transcriptome analysis of bacteria treated with PBT2 and ZnSO₄ for GAS (4.75 μM PBT2+128 μM ZnSO₄), MRSA (2 μM PBT2+50 μM 28 ZnSO₄) and VRE (1.75 μM PBT2+128 μM ZnSO₄) in CAMHB. Genes with log 2 fold changes of >1/<1 and P<0.05 are shown above and below the dashed lines, genes of interest are indicated. Data were collected from 3 biological replicates.

FIG. 2b graphically illustrates the transcript levels for selected genes measured by real-time PCR. Log(2) fold changes were calculated relative to untreated controls and normalised to a reference gene using the ΔΔCt method (reference genes: proS for GAS, rrsA for MRSA, 23S for VRE). Error bars represent standard deviation of 3 biological replicates.

FIG. 2c illustrates the intracellular zinc(II) ion concentrations as determined by ICP MS for GAS and MRSA grown with or without PBT2 and zinc(II) ions (GAS: 0.3 μM PBT2+50 μM ZnSO₄); MRSA: 1 μM PBT2+100 μM ZnSO₄).

FIG. 3: PBT2 and zinc reverse antibiotic resistance in a murine wound infection model. The figure graphically shows CFUs recovered 4 days after wound infection with GAS. Mice were treated twice daily with ointment only or ointment containing 2 mM PBT2, 25 mM ZnSO₄ and/or 1.5% tetracycline. Values for individual mice are plotted. Data is representative of two independent experiments (*=P<0.05, ***=P<0.001, unpaired t44 test, non-parametric).

FIG. 4: Systemic (i.p.) infection model using 1.4×10⁵ CFU colistin resistant K. pneumoniae strain 52.145ΔmgrB at 0 h time point. I.P. treatment doses: PBT2 1.67 mg/kg; Colistin 0.05 mg/kg. The treatment regimen is indicated by the arrows.

FIG. 5: Development of resistance in K. pneumoniae strain MS6671 during serial passage in the presence of sub-inhibitory concentrations of antimicrobial compounds in cation adjusted Mueller Hinton broth. Data is representative of 3 biological replicates.

### DETAILED DESCRIPTION OF THE PREFERRED EMBODIMENTS

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The terms “individual”, “patient” and “subject” are used interchangeably herein to refer to individuals of human or other animal origin and includes any individual it is desired to examine or treat using the methods of the invention. However, it will be understood that these terms do not imply that symptoms are present. Suitable animals that fall within the scope of the invention include, but are not restricted to, humans, primates, livestock animals (e.g., sheep, cows, horses, donkeys, pigs, poultry), laboratory test animals (e.g., rabbits, mice, rats, guinea pigs, hamsters), companion animals (e.g., cats, dogs) and captive wild animals (e.g., foxes, deer, dingoes, birds, reptiles). In some embodiments the individual is a human.

As used herein, the terms “treatment”, “treating”, and the like, refer to administering an agent to obtain a desired pharmacologic and/or physiologic effect. The effect may be prophylactic in terms of completely or partially preventing a disease or symptom thereof and/or may be therapeutic in terms of effecting a partial or complete cure for a disease and/or symptoms of the disease. The effect may be therapeutic in terms of a partial or complete cure for a disease or condition (e.g., a disease or condition mediated by bacterial infection) and/or adverse effect attributable to the disease or condition. These terms also cover any treatment of a condition or disease in a mammal, particularly in a human, and include: (a) preventing the disease or condition or a symptom of a disease or condition from occurring in a subject which may be predisposed to the disease or condition but has not yet been diagnosed as having it (e.g., including diseases or conditions that may be associated with or caused by a primary disease or condition; (b) inhibiting the disease or condition, i.e., arresting its development; (c) relieving the disease or condition, i.e., causing regression of the disease or condition; (d) relieving a symptom of the disease or condition and/or (e) reducing the frequency of a symptom of the disease or condition.

When used herein, the term “pharmaceutically acceptable derivative” includes pharmaceutically acceptable salts or solvates. The term may also include in-vivo hydrolysable esters.

Pharmaceutically acceptable salts are described in, for example, Handbook of Pharmaceutical Salts: Properties, Selection, and Use; Edited by P. Heinrich Stahl and Camile G. Wermuth. VHCA, Verlag Helvetica Chimica Acta, Zürich, Switzerland, and Wiley-VCH, Weinheim, Germany. 2002. Their methods of preparation are well known in the art.

Pharmaceutically acceptable acid addition salts may be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. For example, an amine group of the compounds of the invention may undergo reaction with an acid, for example hydrochloric acid, to form an acid addition salt, for example a hydrochloride or a dihydrochloride.

Pharmaceutically acceptable base addition salts may be prepared from inorganic and organic bases. Corresponding counterions derived from inorganic bases include the sodium, potassium, lithium, ammonium, calcium and magnesium salts. Organic bases include primary, secondary and tertiary amines, substituted amines including naturally-occurring substituted amines, and cyclic amines, including isopropylamine, trimethyl amine, diethylamine, triethylamine, tripropylamine, ethanolamine, 2-dimethylaminoethanol, tromethamine, lysine, arginine, histidine, caffeine, procaine, choline, betaine, ethylenediamine, glucosamine, N-alkylglucamines, theobromine, purines, piperazine, piperidine, and N-ethylpiperidine. For example, where the compound of the invention possesses a carboxylic acid group or a phenol group, the compound may undergo reaction with a base to form the base addition salt.

The term “solvate” is a complex of variable stoichiometry formed by a solute (in this invention, a zinc(II) salt, a zinc ionophore, or a zinc(II) coordination complex) and a solvent. Such solvents should preferably not interfere with the biological activity of the solute. Solvents may be, by way of example, water, acetone, ethanol or acetic acid. Methods of solvation are generally known within the art. In some embodiments, a solvate is pharmaceutically acceptable. In some embodiments, a solvate is a hydrate, for example a mono-, di- or tri-hydrate.

When used herein, the term “pharmaceutically acceptable zinc(II) salt” refers to salts of Zn²⁺. Examples of pharmaceutically acceptable zinc(II) salts include zinc chloride, zinc acetate and zinc sulfate. Other pharmaceutically acceptable zinc(II) salt anions include bromide, phosphate, tosylate, mesylate, tartrate, citrate, succinate, malate, and maleate. The zinc(II) salt may be in the form of a pharmaceutically acceptable solvate, such as a hydrate, for example a mono-, di- or trihydrate. In some embodiments, the combination of zinc(II) salt and zinc ionophore comprises a stoichiometric excess of zinc(II) salt.

When referred to herein, the term “pharmaceutically acceptable derivative” when used in respect of the zinc(II) coordination complexes or zinc ionophores described herein includes, but is not limited to, pharmaceutically acceptable solvates, for example hydrates such as mono-, di- and tri-hydrates; and salts, for example, pharmaceutically acceptable cation salts, anion salts or acid addition salts. Salt derivatives may also form solvates, for example hydrates.

When referred to herein, the term “pharmaceutically acceptable carrier, excipient or diluent” is a solid or liquid filler, diluent or encapsulating substance that can be safely used in systemic administration. Suitable pharmaceutically acceptable carriers, excipients and diluents are well known in the art.

When referred to herein, the term “adjuvant” refers to a pharmacological agent that alters or improves the efficacy of another pharmacologically active agent. An adjuvant is delivered in addition to the primary pharmacologically active agent to enhance its effectiveness. The term “antibiotic adjuvant” refers to an agent that alters or improves the efficacy of an antibiotic. When used herein, the term “potentiator” refers to a reagent that enhances or increases the effect of an antibiotic.

The terms “antibiotic” and the like when used herein refer to a chemical substance used in medicine that is capable of destroying or weakening or inhibiting growth or reducing growth of certain microorganisms that cause infections or infectious diseases, especially pathogenic bacteria. Antibiotics may have activity against one or more classes of bacteria, for example, one or both of Gram-positive and Gram-negative pathogenic bacteria, and find application in the treatment of a wide range of bacterial infections.

In some embodiments, antibiotics of the present invention include, but are not limited to, those having obtained marketing authorisation or regulatory approval, and pharmaceutically acceptable salts, solvates or in vivo hydrolysable esters thereof.

Antibiotics are generally classified according to their mode of action and/or chemical class and/or types of infections that they treat. Classes and examples of antibiotics for treatment of bacterial infection include:
Aminoglycosides
e.g. kanamycin A, amikacin, tobramycin, dibekacin, gentamicin, sisomicin, netilmicin, neomycins B, C, E and streptomycin.
Carbapenems
e.g. ertapenem, doripenem, imipenem, meropenem
Cephalosporins (first generation)
e.g. cefadroxil, cephazolin, cefalotin, cefalexin
Cephalosporins (second generation)
e.g. cefaclor, cefamandole, cefoxitin, cefprozil, cefuroxime
Cephalosporins (third generation)
e.g. cefixime, cefdinir, cefditoren, cefoperazone, cefotaxime, cefpodoxime, ceftazidime, ceftibuten, ceftizoxime, ceftriaxone, cefepime, ceftaroline fosamil,
Cephalosporins (fourth generation)
e.g. cefepime
Cephalosporins (fifth generation)
e.g. ceftaroline fosamil, ceftobipriole
Glycopeptides
e.g. teicoplanin, vancomycin, telavancin, dalbavancin, oritavancin
Lincosamides
e.g. clindamycin, lincomycin
Lipopeptides
e.g. daptomycin
Macrolides
e.g. azithromycin, clarithromycin, dirithromycin, erythromycin, roxithromycin, troleandomycin, telithomycin, spiramycin,
Monobactams
e.g. aztreonam
Nitrofurans
e.g. furazolidine, nitrofurantoin
Oxazolidinones
e.g. linezolid, posizolid, radezolid, torezolid
Penicillins
e.g. amoxicillin, ampicillin, azlocilin, carbenicillin, cloxacillin, dicloxacillin, flucloxacillin, mezlocillin, methicillin, nafcillin, oxacillin, penicillin G, penicillin V, piperacillin, temocillin, ticarcillin
Polypeptides
e.g. bacitracin, colistin (polymyxin E), polymyxin B
Quinolones/fluoroquinolones
e.g. ciprofloxacin, enofloxacin, gatifloxacin, gemifloxacin, levofloxacin, lomefloxacin, moxifloxacin, nalidixic acid, norfloxacin
Sulfonamides
e.g. mafenide, silfacetaminde, sulfadiazine, silver sulfadiazine, sulfadimethoxazine, sulfamethizole, sulfasalazine, sulfisoxazole
Tetracyclines
e.g. demecolcyclin, doxycycline, minicyclin, oxytetracyclin, tetracycline, tigecycline
Other antibiotics
e.g. chloramphenicol, fosfomycin, fusidic acid, metronidazole, mupirocin, thiamphenicol, tigecycline, tinidazole, trimethoprim, rifampicin, rifapentine, pyrazinamide, isoniazid, ethionamide, ethambutol, cycloserine, dapsone, clofazimine.

These antibiotics are named according to their international non-proprietary name (INN). It will be appreciated that each antibiotic mentioned above will also have an IUPAC chemical name in accordance with its chemical structure, and may also have one or more proprietary or brand names. An antibiotic may be in the form of a pharmaceutically acceptable derivative such as a salt, solvate or in vivo hydrolysable ester.

When referred to herein, the term “pharmaceutically acceptable derivative” when used in respect of antibiotics described herein includes, but is not limited to, pharmaceutically acceptable salts, for example cation salts such as sodium or potassium; anion salts such as chloride, acetate, sulphate, methanesulfonate, bromide, and the like; acid addition salts such as hydrochloride; pharmaceutically acceptable solvates, for example hydrates; and esters, for example in vivo hydrolysable esters. In vivo hydrolysable esters are esters which hydrolyse after administration to the subject to provide the free carboxylate group, such as pivaloyloxymethyl ester. Suitable pharmaceutically acceptable derivatives of antibiotics are well known in the art.

Preferably an antibiotic of the present invention is not an aminoglycoside antibiotic and antibiotics of the invention may be referred to herein as a “non-aminoglycoside” antibiotic. In some embodiments the antibiotic is not kanamycin A, amikacin, tobramycin, dibekacin, gentamicin, sisomicin, netilmicin, neomycins B, C, E or streptomycin. In some embodiments the antibiotic is not amikacin. In some embodiments the antibiotic is not a tetracycline antibiotic.

In some embodiments, the antibiotic is a polypeptide antibiotic, for example bacitracin; or a polymyxin, for example colistin (polymyxin E) or polymyxin B. It will be appreciated that the scope of the invention includes other polypeptide antibiotics, including other polymyxin antibiotics. Polymyxin antibiotics are cationic polypeptide antibiotics well known in the art. They are primarily used for gram-negative infections. The present invention includes pharmaceutically acceptable derivatives of polymyxin antibiotics, such as salts and/or solvates, for example anion addition salts, sulfate derivatives, or methane sulfonate derivatives. In some embodiments, a derivative of colistin is an anionic derivative in the form of colistin methane sulfonate, for example in the form of colistin methane sulfonate sodium (colistimethate sodium [CMS]). In some embodiments, colistin is a cationic derivative in the form of colistin sulfate. In some embodiments, colistin or a pharmaceutically acceptable derivative thereof, is administered by an oral, inhalation or topical route, or by parenteral or intravenous route. In some embodiments, a derivative of polymyxin B is polymyxin B sulfate. Polymyxin B, or a pharmaceutically acceptable derivative thereof, in some embodiments, is administered by topical, intramuscular, intravenous, intrathecal or ophthalmic routes.

In some embodiments the antibiotic is a tetracycline antibiotic or a pharmaceutically acceptable derivative thereof. Tetracycline antibiotics include tetracycline, oxytetracycline doxycycline or minocycline, or tigecycline, for example tetracycline. Tetracyclines are broad-spectrum antibiotics, having activity against Gram-positive and Gram-negative bacteria. In some embodiments, tetracycline is administered orally or parenterally.

When used herein the term “ionophore”, unless otherwise stated, refers to a chemical moiety, for example an organic compound, that reversibly binds to ions, for example metal ions. Examples of ionophores include lipid soluble moieties that transport ions, for example metal cations, across a cell membrane. Preferably, the ionophore is pharmaceutically acceptable. In some embodiments the ionophore maybe in the form of a pharmaceutically acceptable derivative or pro-drug.

When used herein the term “zinc ionophore” or “zinc(II) ionophore” unless otherwise indicated refers to an ionophore that reversibly binds to a zinc(II) ion. Preferably, a zinc ionophore is an organic compound. Organic compounds that act as Zinc ionophores are well known in the art, and are commercially available or may be synthesised according to known routes. It will be appreciated that a zinc ionophore may be capable of binding with other metal ions. Examples of pharmaceutically acceptable Zinc(II) ionophores include pyrithione [1-hydroxy-2(1H)-pyridinethione] and substituted 1-hydroxy-2(1H)-pyridinethiones. In another embodiment, pharmaceutically acceptable zinc(II) ionophores include 8-hydroxyquinolines, such as clioquinol (5-chloro-7-iodo-quinolin-8-ol or 5-chloro-7-iodo-8-quinolinol) and PBT2 (5,7-dichloro-2-[(dimethylamino)methyl[quinolin-8-ol or 5,7-dichloro-2-[(dimethylamino)methyl]-8-quinolinol) as described in WO 2004/007461 and US 20080161353 A1. Further zinc(II) ionophores include 8-hydroxyquinoline analogues and derivatives, such as a compound comprising two fused 6-membered rings with a nitrogen at least at the 1-position and a hydroxy substituent at the 8-position. It will be appreciated that the zinc(II) ionophores may be in the form of a pharmaceutically acceptable derivative.

In some embodiments, the compositions, methods and uses of the present invention involve use of a zinc ionophore in the absence of a zinc(II) ion or a zinc(II) salt. The skilled person will understand that zinc ions may be naturally present in a biological system, such as in a human or animal body, or a bacterium. When used herein, references to the absence of zinc(II) ions or zinc(II) salt is intended to mean the absence of any zinc(II) ions, optionally in the form of a zinc(II) salt, additional to what may already be present in the biological system.

Examples of zinc ionophores are disclosed, and the synthesis described in, for example, WO 2017/053696, WO 2016/086261, WO 2014/163622; WO 2010/071944, WO 2007/147217; WO 2007/118276; WO 2005/095360; WO 2004/031161; and WO 2004/007461, each of which is incorporated herein by reference in its entirety. Zinc ionophores may be in the form of a pharmaceutically acceptable derivative.

In some embodiments, the zinc ionophore is clioquinol or PBT2. PBT2 (5,7-dichloro-2-[(dimethylamino)methyl]-8-quinolinol) has undergone phase II clinical trials for treatment Alzheimer's disease and Huntington's disease. Up to 250 mg/day (oral) of PBT2 for 12 weeks has been found to be safe and well tolerated in humans, see, e.g. Lannfelt, L. et al. Safety, efficacy, and biomarker findings of PBT2 in targeting Abeta as a modifying therapy for Alzheimer's disease: a phase IIa, double-blind, randomised, placebo-controlled trial. Lancet Neurol 7, 779-786, doi:10.1016/S1474-4422(08)70167-4 (2008); Huntington Study Group Reach, H. D. I. Safety, tolerability, and efficacy of PBT2 in Huntington's disease: a phase 2, randomised, double-blind, placebo-controlled trial. Lancet Neurol 14, 39-47, doi:10.1016/S1474-4422(14)70262-5 (2015); Bush, A. I. The metal theory of Alzheimer's disease. J Alzheimers Dis 33 Suppl 1, S277-281, doi:10.3233/JAD-2012-129011 (2013). In some embodiments, the zinc ionophore is RA-HQ-12 (5,7-dibromo-2[(4-fluorophenylamino)methyl]-8-quinolinol).

When used herein the terms “pathogenic bacteria” or “pathogenic bacterium” to bacteria that can cause infection. In some embodiments the bacteria are human pathogenic bacteria and cause disease in humans. Bacteria may be classified as gram-positive or gram-negative bacteria according to the structure of the cell wall. Bacteria of the Genus Mycobacterium and related bacteria may be incorporated into the acid-fast group of bacteria. Members of the Genus Mycoplasma and related bacteria do not contain a cell wall and are considered to encompass another distinct group which also contains bacterial pathogenic bacteria.

Gram-positive bacteria include bacilli such as Actinomyces spp.; Bacillus spp.; Corynebacterium spp., Clostridium spp., Lactobacillus spp.; Listeria spp.; coccus such as Streptococcus spp., including S. pyogenes, S. pneumoniae; Enterococcus spp.; Streptomyces spp.; and Staphylococcus spp.; including S. aureus. Examples of antibiotic resistant bacteria include Group A Streptococcus (GAS), vancomycin-resistant Enterococcus (VRE) and methicillin-resistant Staphylococcus aureus (MRSA).

Gram-negative bacteria include gram-negative bacilli including, but not limited to, Acinetobacter baumannii, Acinetobacter lwoffii, Pseudomonas aeruginosa, Pseudomonas fluorescens, Bordetella pertussis, Burkholderia spp., and Sphingobacterium spp.; Enterobacteriacae including Citrobacter spp., Enterobacter spp., Escherichia coli, Klebsiella spp., Morganella spp., Proteus spp., Shigella spp. and Serratia marcescens; and Gram-negative cocci and coccobacilli including Brucella spp., Haemophilus spp. and Neisseria spp.

In some embodiments bacteria include tetracycline- and erythromycin-resistant GAS; multidrug resistant MRSA; multi-drug resistant VRE; and colistin resistant Klebsiella and E. coli.

Examples of bacterial strains include tetracycline- and erythromycin-resistant GAS strain HKU16; multidrug resistant MRSA USA300; multi-drug resistant VRE RBWH1; Klebsiella pneumoniae MS6771; and MCR1-positive E. coli strain MS8345.

### Methods of the Invention

In one aspect the present invention provides the use of a zinc(II) salt in combination with a zinc ionophore for restoring the sensitivity of at least one strain of resistant pathogenic bacteria to an antibiotic; wherein the antibiotic is not an aminoglycoside antibiotic.

In some embodiments, the zinc ionophore is a compound of formula I:

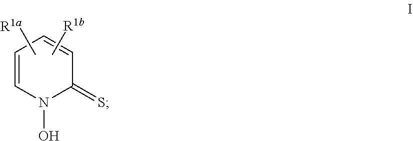

wherein:
R^{1a} and R^{1b} are independently H, halogen, OR^{2a}, SR^{2a}, CF₃, optionally substituted C₁₋₄alkyl, or NR^{2a}R^{2b};
R^{2a} and R^{2b} are independently H, or optionally substituted C₁₋₄alkyl;
or a pharmaceutically acceptable derivative thereof.

In some embodiments, R^{1a} and R^{1b} are both H.

In some embodiments, R^{2a} and R^{2b} are the same. In some embodiments R^{2a} and R^{2b} are both C₁₋₄alkyl.

In some embodiments, the zinc ionophore comprises a compound comprising two fused 6-membered rings with a nitrogen at least at the 1-position and a hydroxy substituent at the 8-position.

In some embodiments, the zinc ionophore is a compound of Formula II:

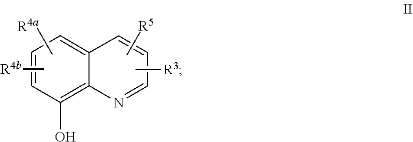

wherein:
R³ and R⁵ are independently H; optionally substituted C₁₋₆ alkyl; optionally substituted C₂₋₆ alkenyl; optionally substituted C₂₋₆alkynyl; optionally substituted C₃₋₆cycloalkyl; optionally substituted aryl; optionally substituted heterocyclyl; CN; OR⁶, SR⁶, COR^{E}, CSR⁶, HCNOR⁶ or HCNNR⁶ in which R⁶ is H, optionally substituted C₁₋₆ alkyl, optionally substituted C₂₋₆alkenyl, optionally substituted C₂₋₆alkynyl, optionally substituted C₃₋₆cycloalkyl, optionally substituted aryl or optionally substituted heterocyclyl; NR⁸R⁹ or SO₂NR⁸R⁹ in which R⁸ and R⁹ are independently selected from H, optionally substituted C₁₋₆ alkyl, optionally substituted C₂₋₆alkenyl, optionally substituted C₂₋₆alkynyl, optionally substituted C₃₋₆cycloalkyl, optionally substituted aryl and optionally substituted heterocyclyl; CONR⁹R¹⁰ in which R⁹ is as defined above and R¹⁰ is optionally substituted C₁₋₆ alkyl, optionally substituted C₂₋₆alkenyl, optionally substituted C₂₋₆alkynyl, optionally substituted C₃₋₆cycloalkyl, optionally substituted aryl or optionally substituted heterocyclyl; CH₂CONR⁸R⁹ in which R⁸ and R⁹ are as defined above; and (CH₂)ₙNR⁹R¹¹ in which R⁹ is as defined above and R¹¹ is selected from optionally substituted C₁₋₆ alkyl, optionally substituted C₂₋₆alkenyl, optionally substituted alkynyl, optionally substituted C₃₋₆cycloalkyl, optionally substituted aryl, optionally substituted heterocyclyl and SO₂R¹² in which R¹² is optionally substituted C₁₋₆alkyl, optionally substituted C₂₋₆alkenyl, optionally substituted C₂₋₆alkynyl, optionally substituted C₃₋₆ cycloalkyl, optionally substituted aryl or optionally substituted heterocyclyl, and n is 1 to 6;
R^{4a} and R^{4b} are independently H, optionally substituted C₁₋₄alkyl or halogen;
or a pharmaceutically acceptable derivative thereof.

In some embodiments, R³ and R⁵ are independently H, optionally substituted C₁₋₆alkyl, CONH₂ or (CH₂)ₙNR⁹R¹¹, wherein n is 0, 1, 2, or 3;

R^{4a} and R^{4b} are independently H, C₁₋₄alkyl or halogen;
R⁹ is H, or optionally substituted C₁₋₄alkyl; and
R¹¹ is optionally substituted C₁₋₄alkyl.

In some embodiments, R³ and R⁵ are independently H, optionally substituted C₁₋₆alkyl, CONH₂ or (CH₂)ₙNR⁹R¹¹, wherein n is 0, 1, 2, or 3;

R^{4a} and R^{4b} are independently H, C₁₋₄alkyl or halogen;
R⁹ is H, or optionally substituted C₁₋₄alkyl; and
R¹¹ is optionally substituted C₁₋₄alkyl.

In some embodiments, R³ and R⁵ are independently H or (CH₂)ₙNR⁹R¹¹, wherein n is 0, 1 or 2, preferably 0 or 1. In some embodiments C₁₋₄alkyl is unsubstituted.

In some embodiments, R^{4a} and R^{4b} are independently H, C₁₋₄alkyl, Cl or I.

In some embodiments, R^{4a} and R^{4b} are independently H, Br, Cl or I. In some embodiments R^{4a} and R^{4b} are independently H, Cl or I.

In some embodiments, the compound of Formula II is a compound of formula III:

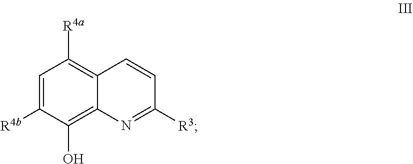

wherein:
R³ is H, optionally substituted C₁₋₆alkyl, CONH₂ or (CH₂)ₙNR⁹R¹¹, wherein n is 0, 1, 2, or 3;
R^{4a} and R^{4b} are independently H, optionally substituted C₁₋₄alkyl or halogen;
R⁹ is H, optionally substituted C₁₋₆ alkyl, optionally substituted C₂₋₆alkenyl, optionally substituted C₂₋₆alkynyl, optionally substituted C₃₋₆cycloalkyl, optionally substituted aryl or optionally substituted heterocyclyl; and
R¹¹ is optionally substituted C₁₋₆ alkyl, optionally substituted C₂₋₆alkenyl, optionally substituted alkynyl, optionally substituted C₃₋₆cycloalkyl, optionally substituted aryl, optionally substituted heterocyclyl or SO₂R¹² in which R¹² is optionally substituted C₁₋₆alkyl, optionally substituted C₂₋₆alkenyl, optionally substituted C₂₋₆alkynyl, optionally substituted C₃₋₆ cycloalkyl, optionally substituted aryl or optionally substituted heterocyclyl;
or a pharmaceutically acceptable derivative thereof.

In some embodiments R³ is CH₂N(C₁₋₄alkyl)₂. In some embodiments R³ is CH₂N(CH₃)₂. In some embodiments the R³ substituent is located on the ring 2-position. In some embodiments R³ is H. In some embodiments, R³ is CH₂NH(4-F—C₆H₄).

In some embodiments R^{4a} and R^{4b} are independently selected from H and halogen. In some embodiments R^{4a} and R^{4b} are independently H, Cl, Br or I. In some embodiments R^{4a} and R^{4b} are independently H, Cl or I; and R³ is H or CH₂N(CH₃)₂. In some embodiments, R^{4a} and R^{4b} are both Br.

For compounds of Formula II or Formula III, optional substituents for the variables R³ or R⁵ refer to one or more groups selected from C₁₋₆ alkyl, C₃₋₆ cycloalkyl, C₂₋₆alkenyl, C₂₋₆ alkynyl, aryl, heterocyclyl, halo, haloC₁₋₆alkyl, haloC₃₋₆cycloalkyl, haloC₂₋₆alkenyl, haloC₂-₆alkynyl, haloaryl, haloheterocyclyl, hydroxy, C₁₋₆ alkoxy, C₂₋₆alkenyloxy, C₂₋₆alkynyloxy, aryloxy, heterocyclyloxy, carboxy, haloalkoxy, haloC₂₋₆alkenyloxy, haloC₂₋₆alkynyloxy, haloaryloxy, nitro, nitroC₁₋₆,alkyl, nitroC₂₋₆alkenyl, nitroaryl, nitroheterocyclyl, azido, amino, C₁₋₆alkylamino, C₂₋₆alkenylamino, C₂₋₆alkynylamino, arylamino, heterocyclylamino acyl, C₁₋₆alkylacyl, C₂₋₆alkenylacyl, C₂₋₆alkynylacyl, arylacyl, heterocyclylacyl, acylamino, acyloxy, aldehydo, C₁₋₆alkylsulphonyl, arylsulphonyl, C₁₋₆alkylsulphonylamino, arylsulphonylamino, C₁₋₆alkylsulphonyloxy, arylsulphonyloxy, C₁₋₆alkylsulphenyl, C₂₋₆ alkylsulphenyl, arylsulphenyl, carboalkoxy, carboaryloxy, mercapto, C₁₋₆alkylthio, arylthio, acylthio, cyano and the like. In some embodiments, the optional substituent is C₁₋₄ alkyl, halo C₁₋₄ alkyl, hydroxy, halo, C₁₋₄ alkoxy or C₁₋₄ alkylacyl.

In some embodiments, the zinc ionophore of Formula II, Formula III, or Formula IV is:

5-chloro-7-iodo-8-quinolinol (clioquinol):

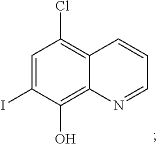 [SMILES: Oc1c(I)cc(Cl)c2cccnc12]

or
5,7-dichloro-2-[(dimethylamino)methyl]-8-quinolinol (PBT2):

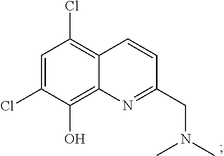 [SMILES: CN(C)Cc1ccc2c(Cl)cc(Cl)c(O)c2n1]

or a pharmaceutically acceptable derivative of either thereof.

In some embodiments, the zinc ionophore is 5,7-dichloro-2-[(dimethylamino)methyl]-8-quinolinol (PBT2) or a pharmaceutically acceptable derivative thereof, for example a hydrochloride addition salt.

In some examples, the zinc ionophore is 5,7-dichloro-2-[(4-fluorophenylamino)methyl]-8-quinolinol (RA-HQ-12):

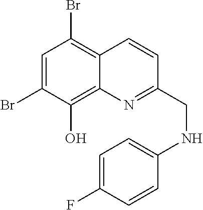 [SMILES: Oc1c(Br)cc(Br)c2ccc(CNc3ccc(F)cc3)nc12]

or a pharmaceutically salt or solvate thereof.

In some embodiments, the zinc ionophore is a compound of Formula IV:

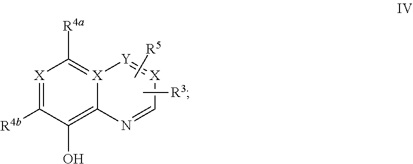

wherein R³, R^{4a}, R^{4b}, R⁵ are as defined above for compounds of Formula II or III;
each X is CH or N;
each Y is CH, CO, CS or N;
or a pharmaceutically acceptable derivative thereof.

In some embodiments, the zinc ionophore is a compound of Formula V wherein the compound of Formula V is a compound of Formula (I) as defined in WO 2004/007461 which is hereby incorporated by reference in its entirety:

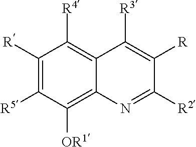

wherein R^{1′} is H, optionally substituted alkyl, optionally substituted alkenyl, optionally substituted acyl, optionally substituted aryl, optionally substituted heterocyclyl, an antioxidant or a targeting moiety;
R is H; optionally substituted alkyl; optionally substituted alkenyl; optionally substituted aryl; optionally substituted heterocyclyl; optionally substituted alkoxy; an antioxidant; a targeting moiety; COR^{6′} or CSR^{6′} in which R^{6′} is H, optionally substituted alkyl, optionally substituted alkenyl, hydroxy, optionally substituted aryl, optionally substituted heterocyclyl, an antioxidant, a targeting moiety, OR^{7′}, SR^{7′} or NR^{7′}R^{8′} in which R^{7′} and R^{8′} are either the same or different and selected from H, optionally substituted alkyl, optionally substituted alkenyl, optionally substituted aryl or optionally substituted heterocyclyl; CN;
(CH₂)ₙNR^{9′}R^{10′}, HCNOR^{9′} or HCNNR^{9′}R^{10′} in which R^{9′} and R^{10′} are either the same or different and selected from H, optionally substituted alkyl, optionally substituted alkenyl, optionally substituted aryl or optionally substituted heterocyclyl and n is 1 to 4; OR^{11′}, SR^{11′} or NR^{11′}R^{12′} in which R^{11′} and R^{12′} are either the same or different and selected from H, optionally substituted alkyl, optionally substituted alkenyl, optionally substituted aryl or optionally substituted heterocyclyl or together form optionally substituted heterocyclyl; or SONR^{13′}R^{14′} in which R^{13′} and R^{14′} are either the same or different and selected from H, optionally substituted alkyl, optionally substituted alkenyl, optionally substituted aryl or optionally substituted heterocyclyl; and
R^{3′}, R^{4′}, R^{5′}, R and R′ are either the same or different and selected from H, optionally substituted alkyl, optionally substituted alkenyl, optionally substituted alkoxy, optionally substituted acyl, hydroxy, optionally substituted amino, optionally substituted thio, optionally substituted sulphonyl, optionally substituted sulphinyl, optionally substituted sulphonylamino, halo, SO₃H, amino, CN, CF₃, optionally substituted aryl, optionally substituted heterocyclyl, an antioxidant or a targeting moiety,
and salts, hydrates, solvates, derivatives, pro-drugs, tautomers and/or isomers thereof.

In some embodiments, the zinc ionophore is a compound of Formula VI wherein the compound of Formula VI is a compound of Formula (I) as defined in WO 2007/147217 which is hereby incorporated by reference in its entirety:

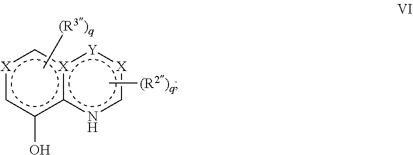

wherein:
R^{2″} is H; optionally substituted C₁₋₆ alkyl; optionally substituted C₂₋₆ alkenyl; optionally substituted C₂₋₆ alkynyl; optionally substituted C₃₋₆ cycloalkyl; optionally substituted aryl; optionally substituted heterocyclyl; CN; OR^{6″}, SR^{6″}, COR^{6″}, CSR^{6″}, HCNOR^{6″} or HCNNR^{6″} in which R^{6″} is H, optionally substituted C₁₋₆ alkyl, optionally substituted C₂₋₆ alkenyl, optionally substituted C₂₋₆ alkynyl, optionally substituted C₃₋₆ cycloalkyl, optionally substituted aryl or optionally substituted heterocyclyl; NR^{8″}R^{9″} or SO₂NR^{8″}R^{9″} in which R^{8″} and R^{9″} are independently selected from H, optionally substituted C₁₋₆ alkyl, optionally substituted C₂₋₆ alkenyl, optionally substituted C₂₋₆ alkynyl, optionally substituted C₃₋₆ cycloalkyl, optionally substituted aryl and optionally substituted heterocyclyl; CONR^{9″}R^{10″} in which R^{9″} is as defined above and R^{10″} is optionally substituted C₁₋₆ alkyl, optionally substituted C₂₋₆ alkenyl, optionally substituted C₂₋₆ alkynyl, optionally substituted C₃₋₆ cycloalkyl, optionally substituted aryl or optionally substituted heterocyclyl; CH₂CONR^{8″}R^{9″} in which R^{8″} and R^{9″} are as defined above; and (CH₂)ₙNR^{9″}R^{11″} in which R^{9″} is as defined above and R^{11″} is selected from optionally substituted C₁₋₆ alkyl, optionally substituted C₂₋₆ alkenyl, optionally substituted alkynyl, optionally substituted C₃₋₆cycloalkyl, optionally substituted aryl, optionally substituted heterocyclyl and SO₂R^{12″} in which R^{12″} is optionally substituted C₁₋₆ alkyl, optionally substituted C₂₋₆alkenyl, optionally substituted C₂₋₆ alkynyl, optionally substituted C₃₋₆ cycloalkyl, optionally substituted aryl or optionally substituted heterocycyl and n is 1 to 6;
R^{x} is independently selected from H, optionally substituted C₁₋₆ alkyl, optionally substituted C₂₋₆ alkenyl, optionally substituted C₂₋₆ alkynyl, optionally substituted C₃₋₆ cycloalkyl, optionally substituted aryl, optionally substituted heterocycyl; optionally substituted C₁₋₆ alkoxy, optionally substituted acyl, hydroxy, optionally substituted amino, optionally substituted thio, optionally substituted sulphonyl, optionally substituted sulphinyl, optionally substituted sulphonylamino, halo, SO₃H, amino, CN, CF₃ and halo;

### X′ is CH or N;

### Y′ is CH, CO, CS or N; and

q is 1, 2 or 3,
and salts, hydrates, solvates, derivatives, pro-drugs, tautomers and/or isomers thereof.

The term “optionally substituted” with regard to compounds of Formula V and VI refers to a group which may or may not be further substituted with one or more groups selected from alkyl, alkenyl, alkynyl, aryl, aldehyde, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy, mercapto, alkylthio, benzylthio, acylthio, cyano, phosphorus-containing groups and the like. Preferably, the optional substituent is C₁₋₆ alkyl, more preferably C₁₋₄alkyl; CF₃; fluorine; chlorine; iodine; cyano; C₁₋₆ alkoxy, more preferably C₁₋₄alkoxy; aryl; heteroaryl; amino; or alkylamino.

As used herein, unless otherwise defined, the term “optionally substituted” typically refers to substitution of a hydrogen atom on a group with a non-hydrogen moiety as detailed below. Any optionally substituted group may bear one, two, three, or more optional substituents.

In some embodiments, the optional substituents are selected from: optionally substituted C₁₋₆alkyl; optionally substituted C₆₋₁₀aryl; halogen; —OH; —NH₂; —NO₂; —SO₂NH₂; —CO₂H; —CO₂(C₁₋₆alkyl); —NHCO₂(C₁₋₆alkyl); —NH—COR^{a} wherein R^{a} is H or C₁₋₆alkyl; —NR^{a}R^{b} wherein R^{a} is H or C₁₋₆alkyl and R^{b} is H or C₁₋₆alkyl; —C(O)NR^{a}R^{b}, wherein R^{a} is H or C₁₋₆alkyl and R^{b} is H, C₁₋₆alkyl; —C(O)R^{a} wherein R^{a} is H or C₁₋₆alkyl; or —Y-Q wherein:

Y is selected from: —O—, —S—, —NH—, —N(C₁₋₆alkyl)-, —NHSO₂—, —SO₂NH—, —NHCONH—, —NHCON(C₁₋₆alkyl)-, —S(O)_{q}- wherein q is 0, 1 or 2, —C(O)NH—, —C(O)N(CH₃)—, —NHC(O)—, —C(O)—, —NHC(NH)NH—, or absent, and
Q is selected from: optionally substituted C₆₋₁₀aryl; optionally substituted 5-10 membered C₁₋₉heteroaryl; optionally substituted 3-10 membered C₁₋₉heterocyclyl; optionally substituted C₃₋₁₀cycloalkyl; optionally substituted C₁₋₆alkyl; optionally substituted C₂₋₆alkenyl; optionally substituted C₂₋₆alkynyl; and hydrogen.

In some embodiments, the optional substituents for an alkyl group are selected from: C₃₋₇cycloalkyl, heterocyclyl, OR, SR, CF₃, CO₂R and halogen; wherein R is selected from H; C₁₋₆alkyl; optionally substituted C₆₋₁₀aryl; optionally substituted 5-10 membered C₁₋₉heteroaryl; optionally substituted 3-10 membered C₁₋₉heterocyclyl; and optionally substituted C₃₋₁₀cycloalkyl.

In some embodiments, the optional substituents for an aryl group are selected from: C₁₋₆alkyl, C₃₋₇cycloalkyl, heterocyclyl, OR, SR, CF₃, CO₂R and halogen; wherein R is selected from H; C₁₋₆alkyl; optionally substituted C₆₋₁₀aryl; optionally substituted 5-10 membered C₁₋₉heteroaryl; optionally substituted 3-10 membered C₁₋₉heterocyclyl; and optionally substituted C₃₋₁₀cycloalkyl.

The term “alkyl” includes saturated aliphatic groups, including straight-chain alkyl groups (e.g., methyl, ethyl, propyl, butyl, pentyl, hexyl, etc.), and branched-chain alkyl groups (isopropyl, tert-butyl, isobutyl, etc.). The expression “C_{x-y}alkyl”, wherein x is 1-2 and y is 2-6 indicates an alkyl group (straight- or branched-chain) containing the specified number of carbon atoms. For example, the term C₁₋₄alkyl includes methyl, ethyl, propyl, butyl, isopropyl, tert-butyl, sec-butyl and isobutyl.

In one embodiment, a straight chain or branched chain alkyl has 6 or fewer carbon atoms (i.e. C₁₋₆). In some embodiments a straight chain or branched chain alkyl has 4 or fewer carbon atoms (i.e. C₁₋₄).

The term “cycloalkyl” includes saturated cyclic aliphatic groups (cyclopropyl, cyclobutyl, cyclopentyl, cyclohexyl, cycloheptyl, cyclooctyl). The term C₃₋₆cycloalkyl includes, but is not limited to, cyclopropyl, cyclopentyl, and cyclohexyl. Likewise, preferred cycloalkyls have from 3-7 carbon atoms in their ring structure, and more preferably have 5 or 6 carbons in the ring structure. As used herein the term “heterocycloalkyl” refers to a cycloalkyl group containing one or more endocyclic heteroatoms.

The term “aryl” refers to aromatic monocyclic (e.g. phenyl) or polycyclic groups e.g., tricyclic, bicyclic, e.g., naphthalene, anthryl, phenanthryl. Aryl groups can also be fused or bridged with alicyclic or heterocyclic rings which are not aromatic so as to form a polycycle. In some embodiments an aryl group is phenyl.

The term “heteroaryl”, as used herein, represents a monocyclic or bicyclic ring, typically of up to 7 atoms in each ring, wherein at least one ring is aromatic and contains from 1 to 4 heteroatoms selected from the group consisting of O, N and S. Heteroaryl groups within the scope of this definition include but are not limited to: benzimidazole, acridinyl, carbazolyl, cinnolinyl, quinoxalinyl, pyrazolyl, indolyl, benzotriazolyl, furanyl, thienyl, benzothienyl, benzofuranyl, quinolinyl, isoquinolinyl, oxazolyl, isoxazolyl, indoiyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom-containing ring, respectively.

The term “heterocycle” or “heterocyclyl” as used herein is intended to mean a 5- to 10-membered aromatic or non-aromatic heterocycle containing from 1 to 4 heteroatoms selected from the group consisting of O, N and S, and includes bicyclic groups. “Heterocyclyl” therefore includes the above mentioned heteroaryls, as well as dihydro and tetrahydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazolidinyl, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom. As referred to herein “heterocycloalkyl” refers to a saturated heterocyclyl group.

The term “ester” includes compounds and moieties that contain a carbon or a heteroatom bound to an oxygen atom that is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above. In vivo hydrolysable esters are esters which hydrolyse after administration to the subject to provide the free carboxylate group. Pro-drugs may be in the form of an in vivo hydrolysable ester.

The term “halogen” includes fluorine, bromine, chlorine, and iodine. The term “perhalogenated” generally refers to a moiety wherein all hydrogens are replaced by halogen atoms, for example CF₃.

The term “heteroatom” includes atoms of any element other than carbon or hydrogen. Preferred heteroatoms are nitrogen, oxygen, and sulfur. In some embodiments heteroatoms are nitrogen, and oxygen.

It is also to be understood that definitions given to the variables of the generic formulae described herein will result in molecular structures that are in agreement with standard organic chemistry definitions and atom valencies.

It will be noted that the structures of some of the compounds of this invention may include asymmetric centres, including asymmetric carbon atoms. It is to be understood accordingly that the isomers arising from such asymmetry (e.g., all enantiomers, stereoisomers, rotamers, tautomers, diastereomers, or racemates) are included within the scope of this invention. Such isomers can be obtained in substantially pure form by classical separation techniques or by stereochemically controlled synthesis. Furthermore, the structures and other compounds and moieties discussed in this application also include all tautomers thereof. Compounds described herein may be obtained through art recognized synthesis strategies. It will also be noted that the substituents of some of the compounds of this invention include isomeric cyclic structures. It is to be understood accordingly that constitutional isomers of particular substituents are included within the scope of this invention, unless indicated otherwise.

In one aspect of the invention, the zinc(II) salt combines with two molar equivalents of the zinc ionophore to form a zinc(II) coordination complex of Formula VII:

Zn(II)[L]₂ VII

wherein each L is the same, and is an anion of a zinc ionophore according to Formula I, Formula II, Formula III, Formula IV, Formula V or Formula VI as hereinbefore defined.

Zinc(II) coordination complexes of Formula VII are referred to as Formula VII^{I} VII^{II}, VII^{III}, VII^{IV}, VII^{V}, or VII^{VI} according to the definition of L.

Accordingly, in some embodiments defined as Formula VII^{I}, L is a ligand of formula IA:

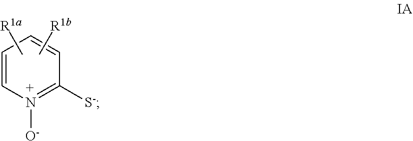

wherein R^{1a} and R^{1b} are as defined above for compounds of Formula I.

In some embodiments defined as Formula VII^{II}, L is a ligand of Formula IIA:

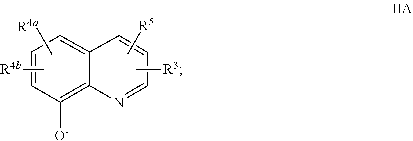

wherein R³, R^{4a}, R^{4b}, and R⁵ are as defined above for compounds of Formula II.

In some embodiments defined as Formula VII^{III}, L is a ligand of Formula IIIA:

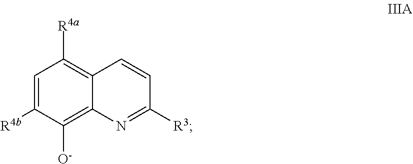

wherein R³, R^{4a}, and R^{4b} are as defined above.

In some embodiments defined as Formula VII^{IV}, L is a ligand of Formula IVA:

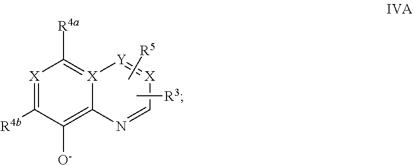

wherein R³, R^{4a}, R^{4b}, R⁵, X and Y are as defined above for compounds of Formula IV.

In some embodiments, defined as Formula VII^{V} or Formula VII^{VI}, L is an anion of a compound of formula V or VI as hereinbefore defined.

In some embodiments, L is [PBT2] or [CQ]:

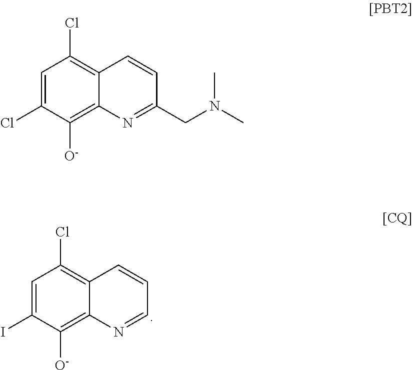 [SMILES: CN(C)Cc1ccc2c(Cl)cc(Cl)c([O-])c2n1 ; [O-]c1c(I)cc(Cl)c2cccnc12]

In some embodiments, L is [RA-HQ-12]:

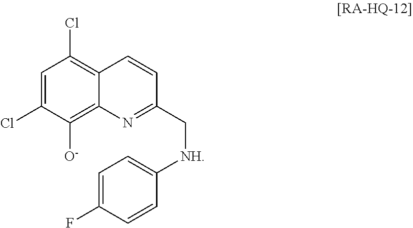 [SMILES: [O-]c1c(Cl)cc(Cl)c2ccc(CNc3ccc(F)cc3)nc12]

In some embodiments, the zinc(II) complex Zn(II)[L]₂ is a complex having the following formula:

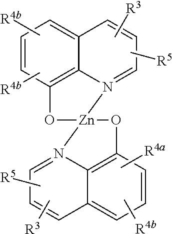

wherein:

R³, R^{4a}, R^{4b}, and R⁵ are as hereinbefore defined for compounds of Formulae II, III, or V;

or a pharmaceutically acceptable derivative thereof.

In some embodiments, the lipophilicity [Log P (octanol:water)] of a zinc(II) complex of Formula VII is less than 5.

It will be noted that some of the zinc(II) complexes of the invention may exist as geometric isomers, for example cis or trans isomers. The zinc(II) complexes of the invention may be in the form of one or other geometric isomer, or a mixture of both. It is to be understood that geometric isomers are included within the scope of this invention.

In some embodiments the zinc(II) complex of Formula VII^{I} is:

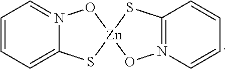

It will be appreciated that this complex may also exist as the geometric isomer:

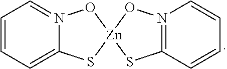

It is to be understood that both geometrical isomers are encompassed by the invention. The isomers may exist singly, or as mixtures of both isomers in any ratio.

In some embodiments the zinc(II) coordination complex of Formula VII is zinc(II)[PBT]₂:

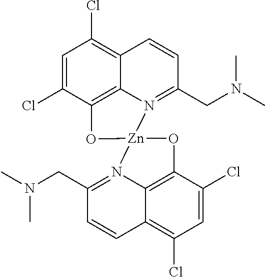

or a pharmaceutically derivative thereof, such as an acid addition salt, for example a hydrochloride addition salt.

References to zinc(II) complexes in the following paragraphs also encompass combinations of a zinc(II) salt and a zinc ionophore, preferably in a molar ratio of approximately 1:2 or 1:1, or in a stoichiometric excess of zinc comprising molar ratios of ionophore:zinc of 1:4 to 1:400.

One or more zinc(II) complexes of the invention, or one or more of combinations of a zinc(II) salt and a zinc ionophore of the invention, are considered to have activity as inhibitors of antibiotic resistance in at least one species of pathogenic bacteria and can restore the sensitivity of the resistant pathogenic bacteria to the antibiotic. They are therefore considered useful in the treatment of bacterial infections, for example in the treatment of one or more bacterial infections caused by antibiotic resistant bacteria. The zinc(II) complexes of the invention are considered useful when administered to a subject in combination with an antibiotic.

Accordingly, the present invention also provides the use of a zinc(II) complex, or a combination of a zinc(II) salt and a zinc ionophore, of the invention as an antibiotic adjuvant or an antibiotic potentiator. A zinc(II) complex of Formula VII may be used as an antibiotic adjuvant or antibiotic potentiator in combination with an antibiotic in the treatment of bacterial infection.

There is also provided a method of treating bacterial infections in a subject which comprises the administration to a patient in need thereof of an inhibitory amount of a zinc(II) complex, or a combination of a zinc(II) salt and a zinc ionophore, as hereinbefore defined, concurrently and/or sequentially with administration of a therapeutically effective amount of an antibiotic or a pharmaceutically acceptable derivative thereof. In some embodiments the bacterial infection is caused by antibiotic resistant bacteria.

In some embodiments, the Zn²⁺/zinc ionophore combination is Zn²⁺/PBT2, or the zinc(II) complex is zinc(II)[PBT2]₂, and one or more of the following apply:
the antibiotic is a polypeptide antibiotic, for example polymyxin B or colistin, and the bacterial infection is caused by a Klebsiella spp., for example Klebsiella pneumoniae; Escherichia coli; an erythromycin-resistant group A Streptococcus (GAS); a methicillin-resistant Staphylococcus aureus (MRSA); or a vancomycin-resistant Enterococcus (VRE);
the antibiotic is tetracycline and the bacterial infection is caused by a Klebsiella spp, for example Klebsiella pneumoniae; an erythromycin-resistant group A Streptococcus (GAS); Streptococcus pneumoniae; or a vancomycin-resistant Enterococcus (VRE);
the antibiotic is tigecycline and the bacterial infection is caused by a Klebsiella spp;
the antibiotic is doxycycline and the bacterial infection is caused by a Klebsiella spp;.
the antibiotic is oxacillin and the bacterial infection is caused by a methicillin-resistant Staphylococcus aureus (MRSA);
the antibiotic is erythromycin and the bacterial infection is caused by a methicillin-resistant Staphylococcus aureus (MRSA);
the antibiotic is ampicillin and the bacterial infection is caused by a methicillin-resistant Staphylococcus aureus (MRSA) or Streptococcus pneumoniae;
the antibiotic is vancomycin and the bacterial infection is caused by a vancomycin-resistant Enterococcus (VRE);
the antibiotic is penicillin and the bacterial infection is caused by Streptococcus pneumoniae;
the antibiotic is chloramphenicol and the bacterial infection is caused by Streptococcus pneumoniae.

In some embodiments, the Zn²⁺/zinc ionophore combination is Zn²⁺/PBT2, or the zinc(II) complex is zinc(II)[PBT2]₂, the antibiotic is a polypeptide antibiotic, for example polymyxin B or colistin, and the bacterial infection is caused by colistin-resistant pathogens, for example a Pseudomonas spp. such as P. aeruginosa or an Acinetobacter spp. such as A. baumannii.

In some embodiments, the Zn²⁺/zinc ionophore combination is Zn²⁺/RA-HQ-12, or the zinc(II) complex is zinc(II)[RA-HQ-12]₂, the antibiotic is a polypeptide antibiotic, for example polymyxin B or colistin, and the bacterial infection is caused by a Klebsiella spp., for example Klebsiella pneumoniae; an erythromycin-resistant group A Streptococcus (GAS); a methicillin-resistant Staphylococcus aureus (MRSA); or a vancomycin-resistant Enterococcus (VRE). In some embodiments, the Zn²⁺/zinc ionophore combination is Zn²⁺/RA-HQ-12, or the zinc(II) complex is zinc(II)[RA-HQ-12]₂, the antibiotic is tetracycline and the bacterial infection is caused by an erythromycin-resistant group A Streptococcus (GAS).

In some embodiments, the Zn²⁺/zinc ionophore combination is Zn²⁺/clioquinol, or the zinc(II) complex is zinc(II)[CQ]₂ and one or more of the following apply:
the antibiotic is a polypeptide antibiotic, for example polymyxin B or colistin, and the bacterial infection is caused by a Klebsiella spp., for example Klebsiella pneumoniae; Escherichia coli, for example MCR-1 E. coli; an erythromycin-resistant group A Streptococcus (GAS), for example Streptococcus pyogenes; a methicillin-resistant Staphylococcus aureus (MRSA); or a vancomycin-resistant Enterococcus (VRE);
the antibiotic is tetracycline and the bacterial infection is caused by a Klebsiella spp, for example Klebsiella pneumoniae; or a vancomycin-resistant Enterococcus (VRE);
the antibiotic is oxacillin and the bacterial infection is caused by a methicillin-resistant Staphylococcus aureus (MRSA);
the antibiotic is erythromycin and the bacterial infection is caused by a methicillin-resistant Staphylococcus aureus (MRSA);
the antibiotic is ampicillin and the bacterial infection is caused by a methicillin-resistant Staphylococcus aureus (MRSA;
the antibiotic is vancomycin and the bacterial infection is caused by a vancomycin-resistant Enterococcus (VRE).

In some embodiments the Zn²⁺/zinc ionophore combination is Zn²⁺/PBT2, or the zinc(II) complex is zinc(II)[PBT2]₂, and one or more of the following apply:
the bacterial infection is caused by a Klebsiella spp., for example Klebsiella pneumoniae, and the antibiotic is polymyxin B, colistin, tetracycline, tigecycline or doxycycline;
the bacterial infection is caused by MCR-1 E. coli, and the antibiotic is polymyxin B or colistin;
the bacterial infection is caused by a vancomycin-resistant Enterococcus (VRE) and the antibiotic is colistin, polymyxin B, tetracycline, or vancomycin;
the bacterial infection is caused by an erythromycin-resistant group A Streptococcus (GAS), for example Streptococcus pyogenes, and the antibiotic is colistin, polymyxin B, or tetracycline;
the bacterial infection is caused by a methicillin-resistant Staphylococcus aureus (MRSA) and the antibiotic is colistin, polymyxin B, oxacillin, ampicillin, or erythromycin;
the bacterial infection is caused by Streptococcus pneumoniae and the antibiotic is tetracycline, penicillin, ampicillin, or chloramphenicol.

In some embodiments the Zn²⁺/zinc ionophore combination is Zn²⁺/clioquinol, or the zinc(II) complex is zinc(II)[CQ]₂ and one or more of the following apply:
the bacterial infection is caused by Streptococcus pneumoniae and the antibiotic is tetracycline, penicillin, ampicillin, or chloramphenicol.
the bacterial infection is caused by Klebsiella pneumoniae and the antibiotic is tetracycline, polymyxin B or colistin;
the bacterial infection is caused by MCR-1 E. coli and the antibiotic is polymyxin B or colistin;
the bacterial infection is caused by erythromycin-resistant group A Streptococcus (GAS), for example Streptococcus pyogenes, and the antibiotic is colistin, or polymyxin B;
the bacterial infection is caused by a methicillin-resistant Staphylococcus aureus (MRSA) and the antibiotic is colistin, polymyxin B, oxacillin, ampicillin, or erythromycin.

In some embodiments, the bacterial infection is caused by tetracycline- and erythromycin-resistant GAS; multidrug resistant MRSA; or VRE.

In some embodiments, the bacterial infection is caused by tetracycline- and erythromycin-resistant GAS strain HKU16; multidrug resistant MRSA USA300; or VRE RBWH1.

In some embodiments, the bacterial infection is caused by resistant Gram-negative Klebsiella pneumoniae MS6771 or MCR1-positive E. coli strain MS8345.

In some embodiments, the bacterial infection is caused by colistin-resistant Gram-negative pathogens such as Pseudomonas aeruginosa strain 253-43-C and Acinetobacter baumannii strain 42-A.

In some embodiments, the antibiotic is a polypeptide antibiotic such as colistin or polymyxin B or a pharmaceutically acceptable derivative of any on thereof, and the Zn²⁺/zinc ionophore combination is Zn²⁺/PBT2. In some embodiments, the antibiotic is a polypeptide antibiotic such as colistin or polymyxin B, and the zinc(II) coordination complex is Zn(II)[PBT2]₂ or a pharmaceutically acceptable derivative of either thereof.

In some embodiments, the antibiotic is a polypeptide antibiotic such as colistin or polymyxin B or a pharmaceutically acceptable derivative of any on thereof, and the Zn²⁺/zinc ionophore combination is Zn²⁺/RA-HQ-12. In some embodiments, the antibiotic is a polypeptide antibiotic such as colistin or polymyxin B, and the zinc(II) coordination complex is Zn(II)[RA-HQ-12]₂ or a pharmaceutically acceptable derivative of either thereof.

In some embodiments, the antibiotic is a polypeptide antibiotic such as colistin or polymyxin B or a pharmaceutically acceptable derivative of either thereof, and the Zn²⁺/zinc ionophore combination is Zn²⁺/clioquinol. In some embodiments, the antibiotic is a polypeptide antibiotic such as colistin or polymyxin B, or a pharmaceutically acceptable derivative of either thereof, and the zinc(II) coordination complex is Zn(II)[CQ]₂ or a pharmaceutically acceptable derivative thereof.

One or more zinc ionophores of the invention, in the absence of a zinc(II) salt or zinc ion source, are considered to have activity as inhibitors of antibiotic resistance in at least one species of pathogenic bacteria and can restore the sensitivity of the resistant pathogenic bacteria to the antibiotic. Zinc ionophores of Formulae I-VI are therefore considered useful in combination with an antibiotic for the treatment of bacterial infections, and preferably in the treatment of one or more bacterial infections caused by antibiotic resistant bacteria.

In a further aspect the present invention also provides the use of a zinc ionophore for restoring the sensitivity of a resistant pathogenic bacteria, preferably a resistant pathogenic gram-negative bacteria, to an antibiotic. In another aspect, there is provided the use of a zinc ionophore for inhibiting resistance of pathogenic bacteria to an antibiotic. The present invention also provides the use of a zinc ionophore as an antibiotic adjuvant or antibiotic potentiator. In certain embodiments, the zinc ionophore is pharmaceutically acceptable. In some embodiments the zinc ionophore is used in combination with an antibiotic for treatment of antibacterial infection. In some embodiments, the zinc ionophore is a compound of formula I, II, III, IV, V, or VI as hereinbefore defined. In some embodiments, the zinc ionophore is clioquinol or PBT2. In some embodiments, the zinc ionophore is RA-HQ-12. The zinc ionophores of the invention are considered useful when administered to a subject in combination with an antibiotic.

There is also provided a method of treating bacterial infections in a subject which comprises the administration to a patient in need thereof of an inhibitory amount of a zinc ionophore as hereinbefore defined, concurrently and/or sequentially with administration of a therapeutically effective amount of an antibiotic, or a pharmaceutically acceptable derivative thereof. In some embodiments the bacterial infection is caused by antibiotic resistant bacteria.

In some embodiments, the antibiotic is not an aminoglycoside antibiotic. In some embodiments, the antibiotic is a polypeptide antibiotic, for example polymyxin B or colistin; and the zinc ionophore is of Formula I-VI, for example an ionophore of Formula III, e.g. clioquinol or PBT2. In some embodiments the resistant pathogenic bacteria is gram negative, for example a Klebsiella spp. or Escherichia coli.

In some embodiments, the antibiotic is a polypeptide antibiotic, for example polymyxin B or colistin or a pharmaceutically acceptable derivative of either thereof, and the adjuvant is a zinc ionophore of Formula I-VI, for example clioquinol or PBT2, and the bacterial infection is caused by Klebsiella spp., for example Klebsiella pneumoniae including MS6771; or Escherichia coli, for example MCR1-positive E. coli including strain MS8345.

In some embodiments, the antibiotic is a polypeptide antibiotic, for example polymyxin B or colistin or a pharmaceutically acceptable derivative of either thereof, and the antibiotic adjuvant or antibiotic potentiator is:
a zinc ionophore of Formula I-VI, for example clioquinol or PBT2, or a pharmaceutically acceptable derivative thereof;
a zinc(II) salt or a pharmaceutically acceptable solvate thereof, in combination with a a zinc ionophore of Formula I-VI, for example clioquinol or PBT2, or a pharmaceutically acceptable derivative thereof; or
a zinc(II) coordination complex of Formula VII, for example Zn(II)[CQ]₂ or

Zn(II)[PBT2]₂.

In some embodiments, the antibiotic is a polypeptide antibiotic, for example polymyxin B or colistin or a pharmaceutically acceptable derivative of either thereof, and the antibiotic adjuvant or antibiotic potentiator is RA-HQ-12 or a pharmaceutically acceptable derivative thereof; a zinc(II) salt or a pharmaceutically acceptable solvate thereof, in combination with RA-HQ-12 or a pharmaceutically acceptable derivative thereof; or Zn(II)[RA-HQ-12]₂.

### Compositions of the Invention

Zinc ionophores of the invention are commercially available or may be prepared by synthetic routes well known in the art.

1-Hydroxypyridine-2-thione (PYT, compound of Formula I, R^{1a}=R^{1b}=H) is commercially available from, for example, Aldrich-Sigma Co LLC. Substituted 1-hydroxypyridine-2-thiones of Formula I can be prepared according to known methods. For example, 1-hydroxypyridine-2-thione compounds substituted by NHalkyl, O-alkyl or S-alkyl on the 6-position can be prepared in accordance with the methods described in WO 2000/067699 and U.S. Pat. No. 5,675,013. 1-Hydroxypyridine-2-thione compounds substituted by alkyl or CF₃ can be prepared from the corresponding 2-bromodihydropyridine by reacting with 3-chloroperoxybenzoic acid followed by treatment with sodium hydrosulfide in accordance with the routes described in, for example, J. Med. Chem., 2014, 57, 16, 7126-7135 and J. Amer. Chem. Soc., 1950, 72(10), 4362-4364. The synthesis of 1-hydroxypyridine-2-thione compounds substituted by OH, SH, O-alkyl or S-alkyl on the 4- and/or 5-ring positions are described in JP 47040057, JP 47040052 and Polish Journal of Chemistry, 2007, 81, 1869.

Clioquinol (5-chloro-7-iodo-8-quinolinol, CQ) is readily available from commercial sources such as Sigma-Aldrich Co LLC.

8-Hydroxyquinoline ionophores of Formula II, III, and V are commercially available from e.g. Sigma-Aldrich Co LLC, or may be synthesised in accordance with known methods, or as described herein. Certain zinc ionophores wherein R^{4a} and R^{4b} are H, alkyl or halogen are commercially available, or may be prepared in accordance with the methods of, for example, J. Med Chem., 1972, 987-989. The synthesis of 8-hydroxyquinoline ionophores wherein R^{4a} and R^{4b} are H or alkyl from commercially available aniline derivatives via a Skraup reaction is described in Organic Synthesis, Coll. Vol. 1, 478 (1941). WO 2014/66506 A2 describes the synthesis of 5-bromo-7-alkyl-8-hydroxyquinoline ionophores from the corresponding 7-alkyl-8-hydroxyquinoline compound using N-bromosuccinimide in tetrahydrofuran. 8-Hydroxyquinoline ionophores wherein R^{4a} and R^{4b} are both H, and the R³ and/or R⁵ substituent is on the ring 2-position and is NH₂, CH₃, CO₂H or CONH₂, are commercially available. 8-Hydroxyquinoline ionophores wherein the R³ and/or R⁵ substituent is on the ring 2-position and is —CH₂NR⁹R¹¹ may be prepared in accordance with the routes described in, for example WO 2017/053696, WO 2016/086261, WO 2010/071944, WO 2007/147217; WO 2007/118276; WO 2005/095360; WO 2004/031161 and WO 2004/007461, and US 2014/296251.

Zinc ionophores of Formula IV, V or VI may be prepared according to methods described in, for example, WO 2007/147217 and WO 2004/007461.

PBT2 was synthesized using the synthetic route described in US 20080161353 A1 (Prana Biotechnology Limited).

RA-HQ-12 was synthesized using the synthetic route described in WO 2017/053696 (University of Florida Research Foundation Incorporated).

Zinc(II) coordination complexes of Formula I may be prepared by known routes from a zinc(II) salt and the desired ligand (ionophore) using conventional methods known in the art; see, for example, Magda D. et al., Cancer Res. 2008 Jul. 1; 68(13): 5318-5325. doi: 10.1158/0008-5472.CAN-08-0601, PMCID: PMC3033660, NIHMSID: NIHMS243995, Synthesis and Anticancer Properties of Water-Soluble Zinc Ionophores.

In general, the zinc(II) complexes of the invention may be prepared by reacting a zinc(II) salt such as zinc(II) chloride, zinc(II) acetate or zinc(II) sulfate with an appropriate amount of the desired zinc ionophore (ligand), generally a stoichiometric excess, in a suitable solvent such as an alcohol, water, acetone, N,N-dimethylformamide or dimethyl sulfoxide. The zinc(II) complex may be isolated by known methods, such as precipitation followed by filtration. The resulting Zn(II) complex can be purified by conventional methods such as recrystallization or chromatography. Ligands may be obtained from, for example, Sigma Aldrich Co LLC, or may be made according to known methods.

Zinc(II) complexes of Formula VII^{I} wherein L is a 1-hydroxypyridine-2-thione (also known as pyrithione or PYT):

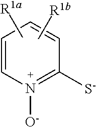

and R^{1a} and R^{1b} are as hereinbefore defined for compounds of Formula I or IA can be prepared by reacting zinc(II) chloride with a 2.5 molar equivalent of the desired pyrithione. For example, Zn[PYT]₂ may be prepared by reacting Zn(II) chloride with a 2.5 molar equivalent of pyrithione in dimethyl sulfoxide as shown in Scheme 1.

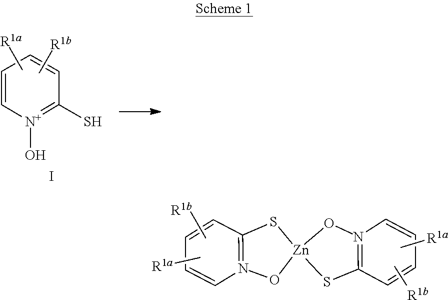

Zinc(II) complexes of Formula VII^{II-VI} can be prepared by reacting one equivalent of a zinc(II) salt, such as zinc(II) chloride or zinc(II) acetate, with two equivalents of the desired ionophore of Formula II, III, IV, V, or VI in methanol or acetone in accordance with methods well known in the art and described in, for example, Magda D. et al., Cancer Res. 2008 Jul. 1; 68(13): 5318-5325. doi: 10.1158/0008-5472.CAN-08-0601, PMCID: PMC3033660, NIHMSID: NIHMS243995, Synthesis and Anticancer Properties of Water-Soluble Zinc Ionophores, as shown in Scheme 2.

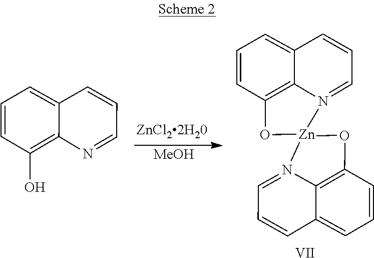

Certain zinc(II) coordination complexes of the invention are considered novel accordingly, in another aspect, the present invention also provides a zinc(II) complex of Formula VII.

The zinc ionophores or zinc(II) complexes of the invention may be in crystalline form. Crystalline zinc(II) complexes or ionophores may exist as polymorphic forms. The zinc(II) complexes or ionophores may also exist in an amorphous form. In some embodiments the zinc(II) complexes or ionophores may be in the form of solvates (e.g. hydrates) and it is intended that these physical forms are within the scope of the present invention. The term “solvate” is a complex of variable stoichiometry formed by a solute (in this invention, a zinc(II) complex or ionophore of the invention) and a solvent. Such solvents should preferably not interfere with the biological activity of the solute. Solvents may be, by way of example, water, acetone, ethanol or acetic acid. Methods of solvation are generally known within the art.

The zinc(II) complexes and zinc ionophores of the present invention may be in the form of a salt, especially a pharmaceutically acceptable acid addition salt. Pharmaceutically acceptable acid addition salts may be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The present invention also provides a pharmaceutical composition comprising an effective amount of a zinc(II) complex or a pharmaceutically acceptable derivative thereof, or an effective amount of a combination of a zinc ionophore or a pharmaceutically acceptable derivative thereof, and a pharmaceutically acceptable zinc(II) salt, as hereinbefore defined, together with at least one pharmaceutically acceptable carrier or diluent.

Antibiotics are readily available from commercial sources such as Sigma-Aldrich Co LLC, or may be synthesized using known methods via fermentation, semi-synthetic or synthetic routes.

The antibiotics referred to herein may be in the form of a pharmaceutically acceptable derivative such as a pharmaceutically acceptable salt, for example a sodium or potassium salt, a chloride, a sulfate, a methanesulfate, or the like, or an in-vivo hydrolysable ester. The antibiotic may also be in the form of a solvate, for example a hydrate. The antibiotic is preferably in a substantially pure form, preferably at least 98% pure on a weight basis.

Pharmaceutically acceptable base addition salts of an antibiotic may be prepared, for example, from inorganic or organic bases. Corresponding counterions derived from inorganic bases include the sodium, potassium, lithium, ammonium, calcium and magnesium salts. Organic bases include primary, secondary and tertiary amines, substituted amines including naturally-occurring substituted amines, and cyclic amines, including isopropylamine, trimethyl amine, diethylamine, triethylamine, tripropylamine, ethanolamine, 2-dimethylaminoethanol, tromethamine, lysine, arginine, histidine, choline, betaine, ethylenediamine, glucosamine, N-alkylglucamines, theobromine, purines, piperazine, piperidine, and N-ethylpiperidine. A carboxylic acid group may undergo reaction with a base to form the base addition salt.

The zinc(II) complexes of the present invention, or a combination of a zinc ionophore and a pharmaceutically acceptable zinc(II) salt, are believed to restore the susceptibility of bacteria to antibiotics by altering the transition metal homeostasis in the bacterial cell.

Accordingly, the compositions, uses and methods of the invention are considered to be useful in the treatment of one or more bacterial infections caused by pathogenic Gram-positive or Gram-negative bacteria which are susceptible to antibiotics.

The compositions, uses and methods of the invention are considered to be effective against resistant bacteria. In some embodiments, the compositions, uses and methods find application in treatment of bacterial infections caused by one or more of a Klebsiella spp., for example Klebsiella pneumoniae; Escherichia coli; an erythromycin-resistant group A Streptococcus (GAS); a methicillin-resistant Staphylococcus aureus (MRSA); or a vancomycin-resistant Enterococcus (VRE).

The compositions, uses and methods of the invention are considered to be effective against diseases or conditions caused by bacterial infections including, and not limited to, septicaemia, pneumonia, bronchiolitis, bronchitis, endocarditis, intra-abdominal infection, joint infection, meningitis, osteomyelitis, pelvic infections, peritonitis, pyelonephritis, and urinary tract infections including cystitis and urethritis.

In the methods of treatment of this invention the zinc ionophore and zinc(II) salt may be administered together, simultaneously, successively or in any order. In some embodiments the zinc ionophore and zinc(II) salt are administered together by the same route. The combination of a zinc ionophore and a pharmaceutically acceptable zinc(II) salt, or a zinc(II) complex, and the antibiotic, may be administered together, simultaneously, successively or in any order. The route of administration of the zinc ionophore and zinc(II) salt, or zinc(II) complex, and the antibiotic may be the same or different. The dose administration regime for the zinc ionophore and zinc(II) salt, or zinc(II) complex, and the antibiotic may be the same or different, and may each be continuous, sequential or sporadic. In some embodiments the components may be administered together as a co-formulation. In some embodiments they may be administered simultaneously or successively in any order via the same, or different, routes of administration.

While it is possible that, for use in therapy, a zinc ionophore and zinc(II) salt, or zinc(II) complex, of the invention may be administered in an undiluted form, however it is preferable to present the zinc(II) complex of Formula I as a pharmaceutical composition.

Thus, in a further aspect of the invention, there is provided a pharmaceutical composition comprising a zinc ionophore and zinc(II) salt, or zinc(II) complex, of the invention and at least one pharmaceutically acceptable carrier, excipient or diluent.

The carrier(s) must be “acceptable” in the sense of being compatible with the other ingredients of the composition and not deleterious to the recipient thereof.

In accordance with the invention, a zinc(II) complex or zinc(II) salt is administered under a therapeutic regime that is non-toxic to the subject. The zinc(II) complex, or zinc(II) salt/zinc ionophore combination, may be administered in unit dose form.

The pharmaceutical compositions of the present invention or the compositions used in the methods of the present invention may be formulated and administered using methods known in the art. Techniques for formulation and administration may be found in, for example, Remington: The Science and Practice of Pharmacy, Loyd V. Allen, Jr (Ed), The Pharmaceutical Press, London, 22^{nd} Edition, September 2012.

The compositions of the invention may be formulated for administration by any route. In some embodiments the composition is formulated for oral administration. An oral formulation may be in the form of tablets, capsules, powders, granules, or liquid preparations. In some embodiments the composition is formulated for topical administration. A topical formulation may be in the form of a cream, lotion, ointment, or gel. In some embodiments the composition is formulated for parenteral administration, for example by an intramuscular, intrathecal, intraperitoneal, intravesical or intravenous route.

The antibiotic is suitably administered with at least one pharmaceutically acceptable carrier in the form of a pharmaceutical composition. In some embodiments an antibiotic is suitably administered parenterally, for example intravenously, intravesicularly or intramuscularly. Accordingly, a suitable composition for administration is an injectable liquid formulation, for example a sterile parenteral solution or suspension. In some embodiments an antibiotic is suitably administered orally.

Suitable unit dosages and maximum daily dosages of antibiotic used in combination with a zinc(II) composition of the invention may be determined in accordance with the unit doses and maximum daily doses used conventionally for a given antibiotic. Accordingly, an antibiotic may be administered to a patient at a daily dosage of, for example, from 250 mg to 750 mg intravenously (IV) or orally every 6 hours to 500 mg to 1 g IV or orally every 6 to 8 hours, with a maximum dose of approximately 50 mg/Kg/day or 4 g/day.

The amount of zinc(II) salt to zinc ionophore administered will vary and can be determined according to the circumstances and the route of administration. In some embodiments the molar ratio of zinc(II) salt to ionophore is approximately 1:2. The amount of zinc(II) complex, or zinc(II) salt and zinc ionophore, to antibiotic administered will vary and can be determined according to the circumstances and the route of administration. The amount of zinc(II) complex, or zinc salt/zinc ionophore, administered should be non-toxic to the subject. In some embodiments the amount of zinc administered is 2 to 100 mg/Kg per day for example: 2.5 to 50 mg/Kg per day; 2.5 to 30 mg/Kg per day; 2.5 to 25 mg/Kg per day or 2.5 to 10 mg/Kg per day per oral. In some embodiments the amount of zinc administered does not exceed 50 mg/Kg per day, for example 20 mg/Kg per day, or 10 mg/Kg per day per oral. It will be appreciated that the ratio of zinc(II) ion to zinc ionophore, or zinc(II) complex, to antibiotic administered will vary, and can be determined according to the circumstances and the route of administration. Furthermore, the ratios for co-administration via the same route may be different to the ratio for administration via separate routes. In some embodiments the molar ratio of antibiotic to zinc(II) ion is from 25:1 to 1:10. In some embodiments the molar ratio of antibiotic to zinc(II) ion (whether in combination with a zinc ionophore or as part of a zinc coordination complex) is from 10:1 to 1:6; 5:1 to 1:5 or 10:1 to 1:1. The ratio of zinc(II) salt to zinc ionophore administered may also vary. In some embodiments, the zinc(II) salt and zinc ionophore is in a ratio of approximately 1:4-4:1, for example 1:2-2:1, or approximately 1:2 or 1:1. In some embodiments, the combination of zinc(II) salt and zinc ionophore comprises a stoichiometric excess of zinc(II) salt, for example, molar ratios of ionophore:zinc of 1:4 to 1:400.

A zinc(II) complex or pharmaceutically acceptable derivative thereof, or a zinc(II) salt and zinc ionophore or pharmaceutically acceptable derivative thereof, as hereinbefore described, may be the sole active ingredient administered to the subject. However in preferred embodiments the zinc(II) complex or zinc(II) salt/zinc ionophore combination is administered with an other therapeutic agent. For example, the zinc composition may be administered with one or more therapeutic agents in combination. The combination may allow for separate, sequential or simultaneous administration of the compound as hereinbefore described with the other active ingredient(s). The combination may be provided in the form of a pharmaceutical composition. Administration with one or more other active ingredients is within the scope of the invention.

In one aspect, a combination of the invention is suitably provided as a kit or commercial package comprising in combination, as active ingredients, a pharmaceutical composition comprising a zinc(II) salt, a zinc ionophore and one or more further pharmaceutical formulations comprising pharmaceutically active ingredients, for example an antibiotic, together with instructions for simultaneous, separate or sequential administration of said combination to a patient in need thereof for use in the treatment of bacterial infection.

In another aspect, a combination of the invention is suitably provided as a kit or commercial package comprising in combination, as active ingredients, a pharmaceutical composition comprising a zinc(II) complex and one or more further pharmaceutical formulations comprising a pharmaceutically active ingredients, for example an antibiotic, together with instructions for simultaneous, separate or sequential administration of said combination to a patient in need thereof for use in the treatment of bacterial infection.

In some embodiments a combination of the invention is a unit dose or fixed dose combination wherein the components of the combination are administered to a patent in the form of a single entity or dosage form.

In some preferred embodiments the zinc(II) complex, or zinc(II) salt and zinc ionophore, is administered with an antibiotic and, optionally, one or more pharmaceutically active ingredients. In some embodiments the antibiotic is colistin, polymyxin B, tetracycline, tigecycline, doxycycline, oxacillin, erythromycin, ampicillin, vancomycin, penicillin, or chloramphenicol. In some embodiments the zinc(II) complex, or zinc(II) salt and zinc ionophore, and antibiotic is administered with one or more further active ingredients selected from, for example: other inhibitors of bacterial resistance, antibiotic potentiators, antibiotics or antibiotic adjuvants, including β-lactamase inhibitors such as clavulanic acid; or other antibiotic adjuvants such as cilastatin, tazobactam, and sulbactam. In some embodiments the zinc(II) complex, or zinc(II) salt and zinc ionophore combination, is administered with one or more antibiotics such as β-lactam antibiotics, for example carbapenems, penicillins or cephalosporins; macrolides such as erythromycin, clarithromycin, or azithromycin; fluoroquinolones such as ciprofloxacin or norfloxacin; sulfonamides such as co-trimoxazole or trimethoprim; tetracyclines such as tetracycline or doxycycline.

As will be readily appreciated by those skilled in the art, the route of administration and the nature of the pharmaceutically acceptable carrier will depend on the nature of the condition and the mammal to be treated. It is believed that the choice of a carrier or delivery system, and route of administration could be readily determined by a person skilled in the art. In the preparation of any formulation containing the compound care should be taken to ensure that the activity of the compound is not destroyed in the process and that the compound is able to reach its site of action without being destroyed. In some circumstances it may be necessary to protect the compound by means known in the art, such as, for example, micro encapsulation or coating (such as the use of enteric coating). Similarly the route of administration chosen should be such that the compound reaches its site of action.

The present invention also contemplates the use of a composition of the invention as a coating on surgical instruments, needles, cannulae, sutures, staples, catheters, stents, artificial joint replacements, and the like as a prophylactic to mitigate against contracting bacterial infection during surgical procedures, intravenous injections, catheterisation etc. In some embodiments there is provided the use of a composition of the invention for coating a catheter.

Those skilled in the art may readily determine appropriate formulations for the compounds of the present invention using conventional approaches. Identification of preferred pH ranges and suitable excipients, for example antioxidants, is routine in the art. Buffer systems are routinely used to provide pH values of a desired range and include carboxylic acid buffers for example acetate, citrate, lactate and succinate. A variety of antioxidants are available for such formulations including phenolic compounds such as BHT or vitamin E, and reducing agents such as methionine or sulfite.

The compounds as hereinbefore described, or pharmaceutically acceptable salt thereof, may be prepared in parenteral dosage forms, including those suitable for intravenous, intrathecal, and intracerebral or epidural delivery. The pharmaceutical forms suitable for injectable use include sterile injectable solutions or dispersions, and sterile powders for the extemporaneous preparation of sterile injectable solutions. They should be stable under the conditions of manufacture and storage and may be preserved against reduction or oxidation and the contaminating action of microorganisms such as bacteria or fungi.

The solvent or dispersion medium for the injectable solution or dispersion may contain any of the conventional solvent or carrier systems for the compound, and may contain, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal and the like. In many cases, it will be preferable to include agents to adjust osmolarity, for example, sugars or sodium chloride. Preferably, the formulation for injection will be isotonic with blood. Prolonged absorption of the injectable compositions can be brought about by the use in the compositions of agents delaying absorption, for example, aluminium monostearate and gelatin. Pharmaceutical forms suitable for injectable use may be delivered by any appropriate route including intravenous, intramuscular, intracerebral, intrathecal, epidural injection, intravesicular administration or infusion. In some embodiments pharmaceutical forms for injectable use may be delivered by intravenous route, or by intravesicular administration by urinary catheter.

Sterile injectable solutions are prepared by incorporating the active compound in the required amount in the appropriate solvent with various of the other ingredients such as those enumerated above, as required, followed by filtered sterilization. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

Other pharmaceutical forms include oral and enteral formulations of the present invention, in which the active compound may be formulated with an inert diluent or with an edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal or sublingual tablets, troches, capsules, elixirs, suspensions, syrups, wafers, and the like. The amount of active compound in such therapeutically useful compositions is such that a suitable dosage will be obtained.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, and a sweetening agent, preservative, dye or flavouring.

Liquid formulations may also be administered enterally via a stomach or oesophageal tube.

Any component used in the preparation of any dosage unit form should be pharmaceutically pure and substantially non-toxic in the amounts employed.

The present invention also extends to any other forms suitable for administration, for example topical application such as creams, lotions and gels; enteral formulations such as suppositories; or compositions suitable for inhalation or intranasal delivery, for example solutions, dry powders, suspensions or emulsions.

Pharmaceutically acceptable vehicles and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It may be advantageous to formulate the compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutically acceptable vehicle. The specification for the novel dosage unit forms of the invention are dictated by and directly dependent on (a) the unique characteristics of the active material and the particular therapeutic effect to be achieved, and (b) the limitations inherent in the art of compounding active materials for the treatment of disease in living subjects having a diseased condition in which bodily health is impaired.

As mentioned above the principal active ingredient may be compounded for convenient and effective administration in therapeutically effective amounts with a suitable pharmaceutically acceptable vehicle in dosage unit form. A unit dosage form can, for example, contain the principal active compound in amounts ranging from 0.25 μg to about 200 mg. Expressed in proportions, the active compound may be present in from about 0.25 μg to about 200 mg/mL of carrier. In the case of compositions containing supplementary active ingredients, the dosages are determined by reference to the usual dose and manner of administration of the said ingredients.

The terms “therapeutically effective amount” and “effective amount” refer to that amount which is sufficient to effect treatment, as defined below, when administered to an animal, preferably a mammal, more preferably a human in need of such treatment. The therapeutically effective amount or effective amount will vary depending on the subject and nature of symptom, disease or condition being treated, the severity of the symptom, disease or condition and the manner of administration, and may be determined routinely by one of ordinary skill in the art.

The invention will now be described with reference to some specific examples and drawings. However, it is to be understood that the particularity of the following description is not to supersede the generality of the invention as hereinbefore described.

### EXAMPLES

### Materials and Methods

### Materials

Zinc sulphate and zinc chloride were purchased from Sigma-Aldrich (Castle Hill, NSW, Australia). The zinc ionophore clioquinol (CQ) was also purchased from Sigma-Aldrich. The zinc ionophores PBT2 and RA-HQ-12 were synthesized by Professor Mark von Itzstein's group (Glycomics Institute, Griffith University, Queensland, Australia). Antibiotics were purchased from Sigma-Aldrich (Castle Hill, NSW, Australia).

### PBT2 and RA-HQ-12 Synthesis

PBT2 was synthesized following the synthetic route below according to US 20080161353 A1.

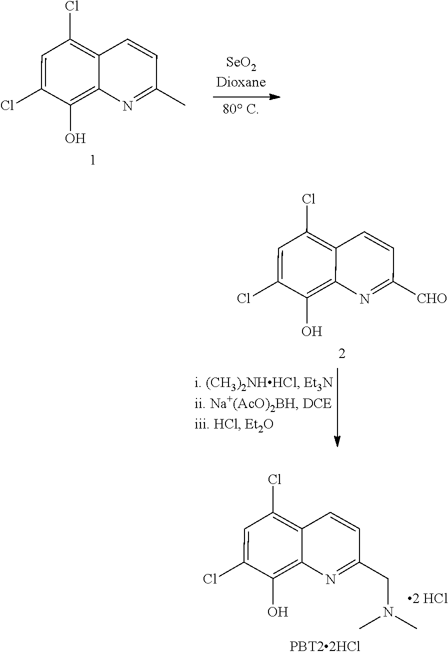 [SMILES: Cc1ccc2c(Cl)cc(Cl)c(O)c2n1 ; O=Cc1ccc2c(Cl)cc(Cl)c(O)c2n1 ; CN(C)Cc1ccc2c(Cl)cc(Cl)c(O)c2n1]

Initially, oxidation of the methyl sidechain of 5,7-dichloro-2-methyl-8-ol (1) was achieved by heating 1 with selenium dioxide in 1,4-dioxane to provide aldehyde 2, in quantitative yield. The resultant crude product was then further reacted with dimethylamine-hydrochloride in 1,2-dichloroethane and triethylamine to yield a product that was reduced in situ, by treatment with sodium triacetoxyborohydride to provide the free amine of PBT2 as an oil. Upon acidification of the free amine with HCl, PBT2 hydrochloride-salt was obtained in 81% yield. The purity was >95% (¹H and ¹³C NMR analysis).

RA-HQ-12 was prepared according to the methods described in WO 2017/053696 (University of Florida Research Foundation Incorporated) at pages 114, 115 and 121.

### General Synthetic Methods

Reagents and dry solvents purchased from commercial sources were used without further purification. Anhydrous reactions were carried out under an atmosphere of argon, using oven-dried glassware. Reactions were monitored using thin layer chromatography (TLC) on aluminium plates pre-coated with Silica Gel 60 F254 (E. Merck). Developed plates were observed under UV light at 254 nm and then visualized after application of a solution of H₂SO₄ in EtOH (5% v/v) and heating. Flash chromatography was performed on Silica Gel 60 (0.040-0.063 mm) using distilled solvents. ¹H and ¹³C NMR spectra were recorded at 400 and 100 MHz respectively on a Bruker Avance 400 MHz spectrometer. Chemical shifts (δ) are reported in parts per million, relative to the residual solvent peak as internal reference [CDCl₃: 7.26 (s) for ¹H, 77.0 (t) for ¹³C]. Low-resolution mass spectra (LRMS) were recorded, in electrospray ionization mode, on a Bruker Daltonics Esquire 3000 ESI spectrometer, using positive ionization mode. The purity of the final product 3 was judged to be >95% by ¹H and ¹³C NMR.

### 5,7-Dichloro-8-hydroxy-2-quinolinecarboxaldehyde (2)

To a stirred suspension of selenium dioxide (1.75 g, 15.80 mmol) in 1,4-dioxane (80 mL) at 55° C. was added a solution of 5,7-dichloro-2-methyl-quinolin-8-ol (2.0 g, 8.77 mmol) in 1,4-dioxane (20 mL) in a dropwise manner over a period of 3 h. After complete addition, the heating temperature was raised to 80° C., and heating was maintained overnight. The reaction mixture was then allowed to cool down to room temperature and the insoluble solids were filtered off on a celite bed. The filtrate was concentrated under vacuum, and the residue was washed with diethyl ether (10 mL×3) to yield 2.10 g (quantitative yield) of the aldehyde 2 as a yellow powder, which was used in the following step without further purification.

### 5,7-Dichloro-2-((N,N-dimethylamino)methyl)quinolin-8-ol HCl salt (3, PBT2 HCl)

To a stirred solution of the crude aldehyde 2 (2.0 g, 8.26 mmol) and dimethylamine hydrochloride (730 mg, 8.96 mmol) in 1,2-dichloroethane (100 mL) was added triethylamine (1.25 mL, 8.96 mmol) in a dropwise manner. The mixture was stirred at room temperature (RT) for 5 min, then sodium triacetoxyborohydride (2.4 g, 11.32 mmol) was added portionwise over a period of 5 min. The mixture was then stirred at RT overnight. Upon reaction completion the reaction mixture was diluted with dichloromethane (200 mL) and washed with saturated sodium bicarbonate (100 mL×3). The organic layer was then dried over anhydrous Na₂SO₄ and concentrated under vacuum to yield an oily product of the free amine base of PBT2. The oily product was triturated with water (100 mL) and extracted with diethyl ether 100 mL x 4). The ethereal extracts were combined, washed with brine, dried over Na₂SO₄ and concentrated under vacuum. To the obtained residue was added 10 mL of concentrated HCl (38% HCl), and the mixture was concentrated in vacuo. The resulting residue was washed with dichloromethane (50 mL×3) to yield 2.30 g of PBT-HCl salt as pale yellow powder (81% yield). ¹H NMR (400 MHz, CDCl₃): δ 2.32 (s, 6H, 2NCH₃), 3.78 (s, 2H, CH₂), 7.51 (s, 1H, H-6), 7.61 (d, J=8.7 Hz, 1H H-4), 8.39 (d, J=8.6 Hz, 1H, H-3); ¹³C NMR (101 MHz, CDCl₃): δ 45.64 (NCH₃), 45.64 (NCH₃), 65.46 (CH₂), 115.51 (C-7), 120.35 (C-5), 122.26 (C-3), 124.07 (q carbon), 127.76 (C-6), 133.83 (C-4), 138.16 (q carbon), 147.98 (C-8), 158.93 (C-2); LRMS [C₁₂H₁₂Cl₂N₂O] (m/z): (+ve ion mode) 272.0 [M+H]⁺.

### Methods

### Bacterial Strains, Media and Growth Conditions

GAS HKU16, MRSA USA300, VRE RBWH1, Klebsiella pneumoniae strain MS6771, E. coli strain MS8345 and Streptococcus pneumoniae strain 23F were grown in Todd-Hewitt broth (THB) or agar with 1% yeast extract (THY) [Todd, E. W. & Hewitt, L. F. A new culture medium for the production of antigenic streptococcal haemolysin. J. Path. Bact. 35, 973-974 (1932)] or in cation-adjusted Mueller-Hinton broth (MHB) [Mueller, J. H. & Hinton, J. A Protein-free Medium for Primary Isolation of the Gonococcus and Meningococcus. Proc. Soc. Exp. Diol. and Med 48, 330-333 (1941)] or agar (supplemented with 2.5% lysed horse blood (LHB) for HKU16 and Streptococcus pneumoniae). Bacteria were routinely grown at 37° C. in ambient air.

Klebsiella pneumoniae strain MS6771 and Streptococcus pneumoniae strain 23F have been previously described (Zowawi H M, Forde B M, Alfaresi M, Alzarouni A, Farahat Y, Chong T M, Yin W F, Chan K G, Li J, Schembri M A, Beatson S A, Paterson D L. Stepwise evolution of pandrug-resistance in Klebsiella pneumoniae. Sci Rep. 2015 5:15082. doi: 10.1038/srep15082; Barnes D M, Whittier S, Gilligan P H, Soares S, Tomasz A, Henderson F W. Transmission of multidrug-resistant serotype 23F Streptococcus pneumoniae in group day care: evidence suggesting capsular transformation of the resistant strain in vivo. J Infect Dis. 1995 171:890-6). E. coli strain MS8345 is a MCR-1 resistant clinical isolate supplied by Prof. D. L. Paterson, University of Queensland Centre for Clinical Research.

P. aeruginosa strain 253-43-C and A. baumannii strain 42-A are colistin resistant clinical isolates. They were obtained from Jan Bell, Australian Centre of Microbial Resistance Ecology (ACARE), University of Adelaide, Australia. The organisms were grown as described above in Todd-Hewitt broth (THB) or agar with 1% yeast extract (THY) or in cation-adjusted Mueller-Hinton broth (MHB).

### Drop Test Assay

Bacteria were diluted from overnight cultures to get a starting OD₆₀₀ of 0.01 in THY. Once the bacteria grew to an OD₆₀₀ of 0.6, the culture were serially diluted in PBS and 5 μL of each dilution (undiluted, 10⁻¹, 10⁻², 10⁻³, 10⁻⁴, 10⁻⁵) was plated on THY agar plates with or without zinc (400 μM) and/or PBT2 (1 μM). Plates were incubated at 37° C. overnight. Drop tests were undertaken in biological triplicates.

### Inductively Coupled Plasma Mass Spectrometry (ICP MS)

Overnight cultures of bacteria were diluted to an OD₆₀₀ of 0.05 in 45 mL MHB (+/−2.5% LHB) and grown to mid-log phase. Cells were harvested at 7,000×g for 7 min at 8° C. The pellet was re-suspended in 20 mL PBS containing 5 mM EDTA and pelleted again at 7,000×g for 7 minutes at 8° C. The wash was repeated two more times and then the pellet was re-suspended in 20 mL PBS without EDTA. The cells were harvested again at 7,000×g for 7 min at 8° C. and then the pellet was washed with PBS. After centrifugation the pellet was re-suspended in 1 mL PBS and transferred into an Eppendorf tube. The cells were pelleted at 18,000×g for 7 min at 8° C., the supernatant was removed and the pellet was dried at 96° C. overnight. The dry pellet was weighed to determine the dry cell weight, re-suspended in 1 mL of 35% HNO₃ and carefully heated to 96° C. for 60 min. After vortexing, the sample was centrifuged at 18,000×g for 25 min to pellet cell debris. For ICP analysis, 200 μL of the supernatant were diluted into 1.8 mL of double distilled H₂O. Samples were analyzed on an Agilent 7500cx ICP-MS (Adelaide Microscopy, University of Adelaide). Biological triplicates were analysed.

### Minimal Inhibitory Concentration (MIC) Determination

MICs were determined by broth microdilution in accordance to CLSI guidelines (“Methods for Dilution Antimicrobial Susceptibility Tests for Bacteria That Grow Aerobically”, Clinical and Laboratory Standards Institute) [Clinical and Laboratory Standards Institute. M100 Performance Standards for Antimicrobial Susceptibility Testing, 27^{th} Edition. (2017)]. MIC assays were undertaken in 96-well plates in a total volume of 100 μL per well. For MRSA, VRE, Klebsiella pneumoniae and E. coli the assays were performed in MHB, and for GAS and Streptococcus pneumoniae MHB+2.5% lysed horse blood (LHB) was used. The bacterial inoculum was prepared by direct colony suspension aiming for 2-8×10⁵ colony forming units (CFU)/mL bacteria per well. Antibiotics/compounds were serially diluted two-fold across the 96-well plate, the last column contained no antibiotics/compounds. The inoculum was added to the plate containing antibiotic/compound and incubated for 16-24 h at 35+/−2° C. The MIC was determined as the lowest concentration of antibiotic/compound that showed no visible growth. MIC assays were carried out in biological triplicates.

### Resistance Development Studies

The development of resistance to antibiotics/compounds was undertaken essentially as previously described [(Ling, L. L. et al. A new antibiotic kills pathogens without detectable resistance. Nature 520, 388, doi:10.1038/nature14303 (2015)]. To investigate resistance development for GAS, MRSA and VRE in the presence of sub-inhibitory concentrations of PBT2 and zinc, bacteria were sequentially passaged over 30 days. As a control, the antibiotic ciprofloxacin was used for GAS and MRSA, and chloramphenicol was used for VRE. Initially, the MIC for PBT2-zinc or antibiotic was determined by broth micro dilution following CLSI guidelines in a microtiter plate. The highest antibiotic or PBT2-zinc concentration that still showed growth after overnight incubation was diluted 1/250 into a new microtiter plate containing two-fold dilutions of antibiotic or PBT2-zinc. This procedure was repeated for 30 days. The assays were undertaken in biological triplicates.

### Bacterial Time-Kill Assays

Bacteria were grown to mid-log phase in THY and then diluted to a starting OD₆₀₀ of 0.05 in THY only or THY containing PBT2 (2 μM for GAS or 6 μM for MRSA and VRE) and/or ZnSO₄ (400 μM for GAS and 600 μM for MRSA and VRE). To determine surviving numbers of bacteria, aliquots were removed at 0, 1, 2, 4, 6 and 24 hours, serial diluted in PBS and plated onto THY agar plates. Viable bacteria were counted after overnight incubation at 37° C. Time-kill assays were performed in biological duplicates.

### Murine Wound Infection Model

For wound infection, 4-7 week old female BALB/c mice were used and housed in individual cages [Pandey, M. et al. A synthetic M protein peptide synergizes with a CXC chemokine protease to induce vaccine-mediated protection against virulent streptococcal pyoderma and bacteremia. J Immunol 194, 5915-5925, doi:10.4049/jimmunol.1500157 (2015)]. The neck area of the mice was shaved and residual hair removed using Nair (Church & Dwight) prior to the experiment. On the day of infection, mice were anesthetized by inhalation of methoxyflurane and a small superficial scarification was made on the shaved skin using a metal file. For infection, 5×10⁶-2×10⁷ CFU GAS or 5×10⁵-2×10⁶ CFU MRSA/VRE were applied onto the scarified skin. After the inoculum had been absorbed by the skin (approximately 10 min) the mice were treated with ointment only (Pharmacy Choice aqueous cream), or ointment containing PBT2 and/or zinc and/or antibiotic (tetracycline for GAS; vancomycin for VRE). Mice were treated with ointment twice daily and a total of 9 treatments were applied. Each treatment consisted of around 25-30 mg ointment and contained 5 mM PBT2 and/or 50 mM ZnSO₄ (MRSA and VRE) or 50 mM ZnCl₂ (GAS). For experiments including antibiotics, the ointment contained 2 mM PBT2 and/or 25 mM ZnSO₄ and/or 1.5% tetracycline or vancomycin. After four days treatment, the mice were euthanized, the scarified skin was excised, and washed twice in PBS by vortexing for 30 seconds. The skin was then homogenized in lysing matrix F tubes using a FastPrep instrument (MP Biomedicals) and plated out on THY plates to determine viable bacteria (for GAS homogenates were plated out on THY with 10 μg/mL neomycin, for MRSA and VRE on THY with 10 μg/mL ampicillin). Each treatment group contained six mice and statistical significance was calculated using an unpaired t-test (non-parametric).

### Murine Systemic Infection Model

For systemic infection, female CD1 mice were infected with 1.4×10*5 CFU of K. pneumoniae strain 52.145ΔmgrB (Kidd et al., A Klebsiella pneumoniae antibiotic resistance mechanism that subdues host defences and promotes virulence, EMBO Mol Med. 2017 April;9(4):430-447. doi: 10.15252/emmm.201607336) via intraperitoneal injection. Four hours post-infection, mice cohorts (n=10) were treated with daily intraperitoneal injections (spanning a period of 3 days). Each treatment consisted of combinations of PBT2 (1.67 mg/kg) and colistin sulfate (0.05 mg/kg) made up in 100 μL of 2% (v/v) DMSO in dH₂O. Negative control mice were administered 100 μL of 2% (v/v) DMSO in dH₂O. Survival was monitored for a period of 5 days.

### RNA Isolation

RNA was isolated using the FastRNA® Pro Blue Kit (MP Biomedicals) and the SV Total RNA Isolation System (Promega). Briefly, bacteria were grown to mid-log phase (OD₆₀₀ 0.4-0.5) in MHB (+2.5% LHB for GAS) in the presence or absence of PBT2 and/or ZnSO₄. Two volumes of RNAprotect (Qiagen) were added to the cultures and the samples were then centrifuged at 5,000×g for 25 min at 4° C. to pellet cells. The dry pellet was stored at −80° C. overnight and then resuspended in 1 mL RNA pro solution (FastRNA® Pro Blue Kit). The sample was transferred to lysing matrix B and processed in a FastPrep instrument (MP Biomedicals). After centrifugation at 13,000×g for 15 min at 4° C., the supernatant was transferred into a fresh tube and incubated at room temperature for 5 min. 300 μl chloroform was added and the mixture vortexed for 10 sec. After 5 min incubation at room temperature, the upper phase was moved to a fresh tube containing 200 μl cold 95% EtOH. The sample was placed on ice for at least 5 min and then transferred into a SV Total RNA Isolation System spin column. The sample was processed according to the manufacturer's instructions and eluted in 110 μl of Nuclease-Free water. To ensure complete removal of DNA, the RNA was then further purified using the TURBO DNA-free kit (Thermo Fisher Scientific) according to the manufacturer's instructions.

### Real-Time PCR

1 μg of isolated RNA was transformed into cDNA using the SuperScriptIII first-strand synthesis kit (Invitrogen). Real-time PCR was undertaken with the SYBR Green Master Mix (Applied Biosystems) following the manufacturer's instructions. Measurements were performed using the ViiA7 real-time PCR system (Life Technologies), using the following conditions: 95° C. for 10 min, 40 cycles of 95° C. for 15 sec, and 60° C. for 1 min, and a final dissociation cycle of 95° C. for 2 min, 60° C. for 15 sec, and 95° C. for 15 sec. Relative gene expression was calculated by the ΔΔCT method using proS (GAS), rrsA (MRSA) and 23S (VRE) as the reference genes. All experiments were done in biological triplicates and measured in technical triplicates. Primers used for real-time PCR are given in Table A (below).

TABLE A
Primers used for real-time PCR

| GAS | | |
|---|---|---|
| proS | Fwd | AGCTGATCTCTGGCGTGAAT |
| proS | Rev | GGGTACGAAGCAAGCCATTA |
| mtsA | Fwd | CAATCGGTCAAGACCCTCAT |
| mtsA | Rev | CCATCAGACACGGCAAAGTA |
| czcD | Fwd | ATTGCACTTTGCACCATGAA |
| czcD | Rev | ACCATCCATCGACCAAACAT |
| glnA | Fwd | TGGATCAGGGATGCACTGTA |
| glnA | Rev | CCCAAGCGACATAAACAGGT |
| copA | Fwd | TCGAAGCTTTGCATCAACTG |
| copA | Rev | GAGCGGAGGTCTGCTATCAC |
| dacC | Fwd | AACACGCCAGCTTATGCTCT |
| dacC | Rev | CCTGATGCTGCCACAAGTAA |
| AdcB | Fwd | ATGGCGGTAGTTGCCATTAG |
| AdcB | Rev | CAAAATCGCCGTTGAAATCT |
| mgA | Fwd | CTGCCGTCTACGACAACAAA |
| mgA | Rev | CCCGTTGGTGAGTCTTGTTT |
| MRSA | | |
| rrsA | Fwd | GAAAGCCACGGCTAACTACG |
| rrsA | Rev | CATTTCACCGCTACACATGG |
| znuC | Fwd | CCGTTTGTCGGAATTGATTT |
| znuC | Rev | TGCTCCTTTGACTAGGGTCAC |
| zntA | Fwd | CGGTGTAAATGATGCACCTG |
| zntA | Rev | TAGCCGAATGCCCAAAATAG |
| copZ | Fwd | GAGCTGTGGTCACTGCAAAA |
| copZ | Rev | CCTTGATCTTCAATTGCGTCT |
| sek | Fwd | CATTTATGGACATAACGGCACT |
| sek | Rev | TTGGTAACCCATCATCTCCTG |
| glnA | Fwd | AAAATGCACGCGGATTTACT |
| glnA | Rev | GGGTTTGCAGCTGGATCTAC |
| frmA | Fwd | TTGGGGATATCAGGTTTTGC |
| frmA | Rev | TCCCGCTAGTTTAGCTCCAA |
| czcD | Fwd | GTTCAGTTGGCGCCATTACT |
| czcD | Rev | ACATGGCAATCATGCACACT |
| VRE | | |
| 23S | Fwd | CTGCATTCCTTAGCCTCCTG |
| 23S | Rev | CTAAGGTTTCCTGGGGAAGG |
| glnA | Fwd | CCGTTATTTGGGATCAATGG |
| glnA | Rev | TAGGCACGAGCATGTTTCAG |
| mntB_2 | Fwd | CGACTGTCGCCGAAATAAAT |
| mntB_2 | Rev | AAAAGCAATGGGGATGAATG |
| copA | Fwd | TCGGAACAAAAATCCCTGAG |
| copA | Rev | AAAAGAATCCGGATGACACG |
| hyl | Fwd | TGGGAAAGAGATGGAGATGG |
| hyl | Rev | AAATAGCTGGCATCGCTGTT |
| ssaB_2 | Fwd | TCTTGGTATTAGCCGGTTGC |
| ssaB_2 | Rev | ATCGCTTGCCTTTTTGATGT |
| zosA | Fwd | ATTGTGCTTCCTTCGTGTCC |
| zosA | Rev | CAGCCGCTTCTTTAGGTGTC |
| dps | Fwd | CCTGCAACAAGTGTTTCCAA |
| dps | Rev | TACATTGCACCCAATGATGG |

### RNASeq Analysis

RNASeq analysis was performed at the Australian Genome Research Facility. The library was prepared using a Ribo-zero stranded protocol. In brief, rRNA was depleted with Ribo Zero, RNA was fragmented (heat and divalent cations) and 1^{st} strand cDNA synthesis was done with SuperScript IT Reverse Transcriptase (Invitrogen). For the 2nd strand cDNA synthesis, the strand was “marked” with dUTP. A 3′ adenylation of DNA fragments was performed followed by sequencing adapter ligation (utilizing T-A pairing of adapter and DNA fragments). The library was amplified by PCR (amplification of “unmarked” 1st strand only). Libraries were assessed using either Agilent's Bioanalyser DNA 1000 chip or TapeStation D1K TapeScreen system. qPCR was used to quantify individual libraries before normalizing (2 nM) and pooling. Libraries were pooled and clustered through the Illumina cBot system using TruSeq PE Cluster Kit v3 reagents followed by sequencing on the Illumina HiSeq 2500 system with TruSeq SBS Kit v3 reagents with 110 (101 read 1, 9 cycles index read). Libraries were sequenced with a HiSeq 2500 ultra-high-throughput sequencing system (Illumina) to produce 100-base-paired end reads. An average of 45 million reads per sample were generated and mapped to the porcine reference genome (Sscrofa10.2) using the 2-pass method of the STAR aligner with default parameters. 80% of these reads uniquely hit to the reference genome. Duplicate reads were denoted with the MarkDuplicates tool of Picard (http://broadinstitute.github.io/picard). Differential gene expression was analysed using Degust (http://victorian-bioinformatics-consortium.github.io/degust/) and figures were generated using R-Studio [RStudio Team. Integrated Development for R. . RStudio, Inc., Boston, Mass. (2015)].

### Sequencing of VRE

To determine the complete genome sequence of RBWH1, long-read, single molecule real-time (SMRT) sequencing was performed using the Pacific Biosciences RS II platform. Genomic DNA was sheared using the HydroShear Plus (Digilab) and a library was prepared using the DNA Template Prep Kit 2.0 (Pacific Biosciences). Sequencing was performed on a single SMRT cell with XL polymerase and Sequencing Kit C2 (Pacific Biosciences). Filtering of the long reads identified 147,593 reads with an average polymerase read length of 4.8 kb. To aid in genome assembly validation, RBWH1 was also sequenced on an Illumina Next-seq to produce paired-end reads with a read length of 150 bases. De novo assembly was performed using Unicycler v0.4. with corrected PacBio long reads generated using the PacBio SMRT analysis v2.3.0. The assembly was manually circularised to generate a chromosomal sequence.

### Potential of PBT2 to Act as an Antibacterial Agent Against GAS Strain HKU16, MRSA Strain USA300, and VRE Clinical Isolate RBWH1

Using Clinical and Laboratory Standards Institute guidelines for antimicrobial sensitivity testing [Clinical and Laboratory Standards Institute. M100 Performance Standards for Antimicrobial Susceptibility Testing, 27th Edition. (2017)], the potential of PBT2 to act as an antibacterial agent was investigated against GAS strain HKU16 (Tse, H. et al. Molecular characterization of the 2011 Hong Kong scarlet fever outbreak. J Infect Dis 206, 341-351, doi:10.1093/infdis/jis362 (2012)], MRSA strain USA300 [Diep, B. A. et al. Complete genome sequence of USA300, an epidemic clone of community-acquired methicillin-resistant Staphylococcus aureus. Lancet 367, 731-739, doi:10.1016/S0140-6736(06)68231-7 (2006)], and VRE clinical isolate RBWH1.

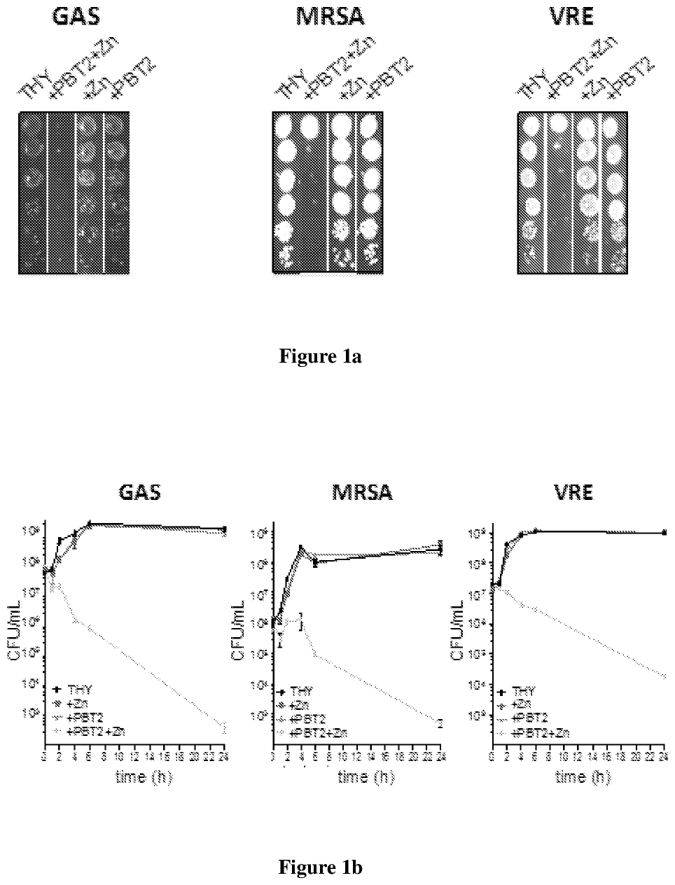

At the concentrations used, neither PBT2 nor zinc(II) ions (in the form of zinc chloride) displayed antibacterial activity. However, the combination of PBT2+zinc chloride exhibited antibacterial activity against each Gram-positive pathogen (FIG. 1A and Table B) and the mode of action was found to be bactericidal in nature (FIG. 1B).

TABLE B
PBT2 and zinc are active against Gram-positive bacterial pathogens

| | GAS HKU 16 | MRSA USA300 | VRE RBWH1 |
|---|---|---|---|
| PBT2 | >30 μM | 3.75 μM | 1.9 μM |
| Zinc(II) ion | 2 mM | 3 mM | 3 mM |
| PBT2 + Zn(II) ion | 4 μM PBT2 + | 1 μM PBT2 + | 1 μM PBT2 + |
| | 800 μM Zn | 800 μM Zn | 800 μM Zn |
| PBT2 + Zn(II) ion | 8 μM PBT2 + | 2 μM PBT2 + | 2 μM PBT2 + |
| | 25 μM Zn | 200 μM Zn | 200 μM Zn |

MIC values were determined by broth micro dilution according to CLSI guidelines, n = 3.

The capacity of each pathogen to develop resistance to the combination of PBT2+zinc chloride was investigated. No resistant mutants of any of the Gram-positive pathogens were identified following serial passage for a period of 30 days in the presence of sub-inhibitory concentrations of PBT2+zinc chloride (FIG. 1C). A wound infection model was used to investigate the efficacy of PBT2+zinc treatment [Pandey, M. et al. A synthetic M protein peptide synergizes with a CXC chemokine protease to induce vaccine-mediated protection against virulent streptococcal pyoderma and bacteremia. J Immunol 194, 5915-5925, doi:10.4049/jimmunol.1500157 (2015)]. The application of PBT2+zinc(II) ions (in the form of either ZnCl₂ or ZnSO₄) resulted in a significant reduction in the bacterial burden at the site of infection (FIG. 1D).

### Changes in the Transcriptome of Each Gram-Positive Pathogen in Response to Sub-Inhibitory Concentrations of PBT2+Zinc(II) Ions

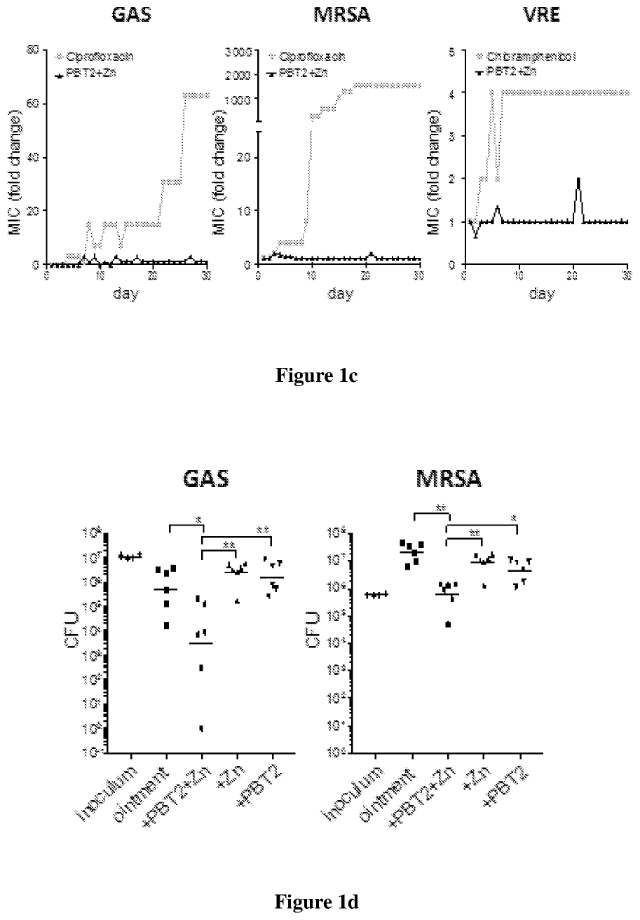

The mechanism of action of treatment with PBT2+zinc(II) ions was investigated by analysis of the transcriptome of each Gram-positive pathogen in response to sub-inhibitory concentrations of PBT2+zinc(II) ions. Changes in the transcription of heavy metal homeostasis genes were observed and confirmed by real-time RT-PCR (FIGS. 2A and 2B).

Although the bacterial transcriptional response to PBT2+zinc(II) ion treatment, included the induction of heavy metal efflux systems, the bacterial intracellular zinc ion concentration was significantly elevated as assessed by inductively-coupled plasma mass spectrometry (FIG. 2C). In addition, the transcription of several essential virulence and metabolic systems was also disrupted by sub-inhibitory concentrations of PBT2+zinc(II) ions (FIGS. 2A and 2B).

### Zinc(II) Ions Resensitize Pathogenic Bacteria Against Various Antibiotic Classes

Given the significant transcriptional changes to bacterial systems induced by the combination of PBT2+zinc(II) ions, whether such disruption enhances antibiotic sensitivity in otherwise resistant bacterial pathogens was investigated using the tetracycline- and macrolide-resistant GAS strain HKU16, and the multidrug-resistant strains MRSA USA300 and VRE RBWH1 and Streptococcus pneumoniae strain 23F. Resistance of the Gram-negative Klebsiella pneumoniae MS6771 and MCR1-positive E. coli strain MS8345 was also examined. In the presence of antibiotic only, MS6771, MS8345, 23F, HKU16, USA300 and RBWH1 exhibited resistance to several antibiotic classes. The addition of either PBT2 or zinc(II) ions alone usually did not affect antibiotic resistance. However, the addition of sub-inhibitory concentrations of PBT2+zinc(II) ions resulted in resistant bacterial strains becoming sensitive to the following antibiotics: Klebsiella pneumoniae MS6771 became sensitive to colistin, polymyxin B, tetracycline, tigecycline and doxycycline, but not to amikacin (Table 1); MCR1-positive E. coli strain MS8345 became sensitive to colistin and polymyxin B (Table 2); Streptococcus pneumoniae strain 23F became sensitive to penicillin, tetracycline and chloramphenicol (Table 3); GAS strain HKU16 became sensitive to tetracycline, polymyxin B and colistin (Table 4); VRE RBWH1 became sensitive to vancomycin, tetracycline, polymyxin B and colistin (Table 5); and MRSA USA300 became sensitive to oxacillin, erythromycin, ampicillin, polymyxin B and colistin (Table 6). Similar results were observed when clioquinol was used in the place of PBT2 in these experiments (Tables 1-6). Additionally, for the Gram-negative pathogens Klebsiella pneumoniae MS6771 and MCR1-positive E. coli strain MS8345, the combination of PBT2 and colistin or PBT2 and polymyxin B also resulted in sensitivity to colistin or polymyxin B (Tables 1-2). Similar results were observed when clioquinol (CQ) was used in the place of PBT2 in these experiments (Tables 1-2).

TABLE 1
MICs for different antibiotics in the presence or absence of Clioquinol
(CQ) or PBT2 and zinc ions for Klebsiella pneumoniae.

| | MIC (μg/mL) | | | |
|---|---|---|---|---|
| | CQ: 0 μM | CQ: 64 μM | | |
| Antibiotic | Zinc: 0 μM | Zinc: 256 μM | CQ: 64 μM | Zinc: 256 μM |
| Colistin | 64 | 0.5 | 0.5 | 32-64 |
| Polymyxin B | 64 | 2 | 2 | 32-64 |
| Chloram- | 128 | 64 | 64 | 128 |
| phenicol | | | | |
| Gentamicin | >128 | >128 | >128 | >128 |
| Tetracycline | 32-64 | 4-8 | 16 | 8 |
| Trimethoprim | >128 | >128 | >128 | >128 |
| Meropenem | 64 | 8-16 | 8-16 | 16-32 |
| Ampicillin | >128 | >128 | >128 | >128 |
| Amikacin | >128 | >128 | >128 | >128 |
| Ceftaroline | >128 | >128 | >128 | >128 |
| Ceftazidime | 32 | 16 | 16 | 32 |
| Cefoxitin | 128 | 64 | 64 | 128 |
| Ciprofloxacin | >128 | >128 | >128 | >128 |
| Tigecycline | 4 | 4 | 4 | 4 |
| Doxycycline | 8-16 | 8 | 8 | 8-16 |

| | MIC (μg/mL) | | | |
|---|---|---|---|---|
| | PBT2: 0 μM | PBT2: 64 μM | | |
| Antibiotic | Zinc: 0 μM | Zinc: 256 μM | PBT2: 64 μM | Zinc: 256 μM |
| Colistin | 32-64 | 0.125 | 0.125 | 32-64 |
| Polymyxin B | 32-64 | 0.125 | 0.125 | 32-64 |
| Chloram- | 128 | 128 | 128 | 128 |
| phenicol | | | | |
| Gentamicin | >128 | >128 | >128 | >128 |
| Tetracycline | 16-32 | 4 | 8 | 8 |
| Trimethoprim | >128 | >128 | >128 | >128 |
| Meropenem | 64 | 16 | 32 | 16-32 |
| Ampicillin | >128 | >128 | >128 | >128 |
| Amikacin | >128 | >128 | >128 | >128 |
| Ceftaroline | >128 | >128 | >128 | >128 |
| Ceftazidime | 32 | 32 | 32 | 32 |
| Cefoxitin | 128 | 64 | 64 | 128 |
| Ciprofloxacin | >128 | >128 | >128 | >128 |
| Tigecycline | 4 | 2 | 2 | 4 |
| Doxycycline | 8-16 | 4 | 4-8 | 8-16 |

MIC values were determined by broth micro dilution according to CLSI guidelines, n = 3.
MIC changes from resistant to sensitive in the presence of PBT2 +/− Zn are highlighted in bold type.

TABLE 2
MICs for different antibiotics in the presence or absence of
Clioquinol (CQ) or PBT2 and zinc ions for MCR-1 E. coli.

| | MIC (μg/mL) | | | |
|---|---|---|---|---|
| | CQ: 0 μM | CQ: 5 μM | | |
| Antibiotic | Zinc: 0 μM | Zinc: 25 μM | CQ: 5 μM | Zinc: 25 μM |
| Polymyxin B | 8 | 1 | 2-4 | 8 |
| Colistin | 8 | 0.5-1 | 2 | 8 |

| | MIC (μg/mL) | | | |
|---|---|---|---|---|
| | PBT2: 0 μM | PBT2: 7 μM | | |
| Antibiotic | Zinc: 0 μM | Zinc: 200 μM | PBT2: 7 μM | Zinc: 200 μM |
| Polymyxin B | 8 | 0.5 | 1 | 8 |
| Colistin | 8 | 0.25 | 1 | 8 |

MIC values were determined by broth micro dilution according to CLSI guidelines, n = 3.
MIC changes from resistant to sensitive in the presence of PBT2 +/− Zn are highlighted in bold type.

TABLE 3
MICs for different antibiotics in the presence or absence
of PBT2 and zinc ions for Streptococcus pneumoniae 23F.

| | MIC (μg/mL) | | | |
|---|---|---|---|---|
| | PBT2: 0 μM | PBT2: 8 μM | | |
| Antibiotic | Zinc: 0 μM | Zinc: 32 μM | PBT2: 8 μM | Zinc: 32 μM |
| Penicillin | 2 | 0.5 | 2 | 2 |
| Ampicillin | 8 | 1 | 8 | 8 |
| Tetracycline | 32 | 1 | 32 | 32 |
| Chloram- | 16 | 2 | 16 | 16 |
| phenicol | | | | |
| Gentamicin* | 64 | 1 | 4 | 64 |

MIC values were determined by broth micro dilution according to CLSI guidelines, n = 3.
MIC changes from resistant to sensitive in the presence of PBT2 +/− Zn are highlighted in bold type.
*No breakpoint information available.

TABLE 4
MICs for different antibiotics in the presence
or absence of Clioquinol (CQ) or PBT2 and zinc ions
for Streptococcus pyogenes strain HKU16.

| | MIC (μg/mL) | | | |
|---|---|---|---|---|
| | CQ: 0 μM | CQ: 6 μM | | |
| Antibiotic | Zinc: 0 μM | Zinc: 80 μM | CQ: 6 μM | Zinc: 80 μM |
| Erythromycin | >128 | >128 | >128 | >128 |
| Tetracycline | 128 | 4 | 128 | 128 |
| Polymyxin B | 64 | 1 | 32 | 64 |
| Colistin | >128 | 1 | 64 | >128 |

| | MIC (μg/mL) | | | |
|---|---|---|---|---|
| | PBT2: 0 μM | PBT2: 4.75 μM | PBT2: 4.75 | Zinc: 128 |
| Antibiotic | Zinc: 0 μM | Zinc: 128 μM | μM | μM |
| Erythromycin | >128 | >128 | >128 | >128 |
| Tetracycline | 64-128 | 2-4 | 64 | 128 |
| Polymyxin B | 64-128 | 1-2 | 32 | 64 |
| Colistin | >128 | 0.5-1 | 32 | >128 |

MIC values were determined by broth micro dilution according to CLSI guidelines, n = 3.
MIC changes from resistant to sensitive in the presence of PBT2 +/− Zn are highlighted in bold type.

TABLE 5
MICs for different antibiotics in the presence or absence
of Clioquinol (CQ) or PBT2 and zinc ions for Vancomycin-resistant
Enterococci (VRE) clinical isolate RBWH1.

| | MIC (μg/mL) | | | |
|---|---|---|---|---|
| | CQ: 0 μM | CQ: 2.5 μM | | |
| Antibiotic | Zinc: 0 μM | Zinc: 32 μM | CQ: 2.5 μM | Zinc: 32 μM |
| Vancomycin | >128 | 1 | >128 | >128 |
| Tetracycline | >128 | 1-2 | 64 | >128 |
| Polymyxin B | 128 | 0.5-1 | 64 | 128 |
| Colistin | >128 | 0.5-1 | >128 | >128 |

| | MIC (μg/mL) | | | |
|---|---|---|---|---|
| | | PBT2: 1.75 | | |
| | PBT2: 0 μM | μM | PBT2: 1.75 | Zinc: 128 |
| Antibiotic | Zinc: 0 μM | Zinc: 128 μM | μM | μM |
| Vancomycin | >128 | 1-2 | >128 | >128 |
| Tetracycline | >128 | 2-4 | 128 | >128 |
| Polymyxin B | 128 | 0.5 | 64 | 128 |
| Colistin | >128 | 0.5 | >128 | >128 |

MIC values were determined by broth micro dilution according to CLSI guidelines, n = 3.
MIC changes from resistant to sensitive in the presence of PBT2 +/− Zn are highlighted in bold type.

TABLE 6
MICs for different antibiotics in the presence or absence
of Clioquinol (CQ) or PBT2 and zinc ions for Methicillin-resistant
Staphylococcus aureus (MRSA) strain USA300.

| | MIC (μg/mL) | | | |
|---|---|---|---|---|
| | CQ: 0 μM | CQ: 0.75 μM | | |
| Antibiotic | Zinc: 0 μM | Zinc: 16 μM | CQ: 0.75 μM | Zinc: 16 μM |
| Methicillin* | >128 | >128 | >128 | >128 |
| Oxacillin* | 128 | 0.5-1 | 128 | 128 |
| | CQ: 0 μM | CQ: 1 μM | | |
| | Zinc: 0 μM | Zinc: 32 μM | CQ: 1 μM | Zinc: 32 μM |
| Erythromycin | 64 | 1 | 64 | 64 |
| Ampicillin | >128 | 1 | >128 | >128 |
| Polymyxin B | 128 | 2 | 64 | 128 |
| Colistin | >128 | 2 | >128 | >128 |
| | MIC (μg/mL) | | | |
| | PBT2: 0 μM | PBT2: 2 μM | | |
| Antibiotic | Zinc: 0 μM | Zinc: 25 μM | PBT2: 2 μM | Zinc: 25 μM |
| Methicillin* | >128 | >128 | >128 | >128 |
| Oxacillin* | 128 | 1-2 | 128 | 128 |
| | PBT2: 0 μM | PBT2: 2 μM | | |
| | Zinc: 0 μM | Zinc: 50 μM | PBT2: 2 μM | Zinc: 50 μM |
| Erythromycin | 64 | 0.5 | 32 | 64 |
| Ampicillin | >128 | 2 | >128 | >128 |
| Polymyxin B | 64 | 2 | 64 | 64 |
| Colistin | >128 | 2 | >128 | >128 |

MIC values were determined by broth micro dilution according to CLSI guidelines, n = 3.
MIC changes from resistant to sensitive in the presence of PBT2 +/− Zn are highlighted in bold type.
*+2% NaCl as per CLSI guidelines.

TABLE 7
MICs for polymyxin antibiotics colistin and polymyxin
B in the presence or absence of PBT2

MIC (μg/mL)

| | | |
|---|---|---|
| Antibiotic | Untreated | PBT2 (16 μL) |

A Baumannii

| | | |
|---|---|---|
| Colistin | >64 | <0.125 |

P. Aeruginosa

| | | |
|---|---|---|
| Polymyxin B | 8 | 1 |
| Colistin | 8 | 1 |

P. aeruginosa strain 253-43-C and A. baumannii strain 42-A MIC assays were undertaken in the absence (untreated) or presence of PBT2.
MIC values highlighted in bold indicate an antibiotic susceptible breakpoint (≤4 μg/mL) in accordance with CLSI guidelines.
Data represents mean of 3 biological replicates.

Efficacy of RA-HQ-12+Zinc(II) Ions in Combination with Antibiotics on Infection

The combination of RA-HQ-12+zinc(II) ions (ZnSO₄) was observed to resensitize GAS HKU16, MRSA USA 300, VRE RBWH1 and Klebsiella pneumoniae MS6671 to the polymyxin class antibiotics colistin and polymyxin B. Intermediate antibiotic resensitization of GAS HKU16 to tetracycline was observed in the presence of RA-HQ-12 and ZnSO₄ (Table 8).

TABLE 8
Combination of RA-HQ-12 and zinc sulphate resensitises gram-positive
and Gram-negative pathogens to polymyxin classes of antibiotics

| | MIC (μg/mL) | | | |
|---|---|---|---|---|
| | GAS | | | |
| | RA-HQ-12: | RA-HQ-12: | | |
| | 0 μM | 3.5 μM | RA-HQ-12: | |
| Antibiotic | Zn: 0 μM | Zn: 128 μM | 3.5 μM | Zn: 128 μM |
| Erythromycin | >128 | >128 | >128 | >128 |
| Azithromycin | >128 | >128 | >128 | >128 |
| Tetracycline | >128 | 8 | 128 | >128 |
| Polymyxin B | 128 | 4^{#} | 128 | 128 |
| Colistin | >128 | 2-4^{#} | 128 | >128 |
| Ampicillin | 0.125 | 0.125 | 0.125 | 0.125 |
| Vancomycin | 0.5 | 0.25 | 0.5 | 0.5 |
| Oxacillin* | 0.125 | 0.125 | 0.125 | 0.125 |
| | MRSA* | | | |
| | RA-HQ-12: | RA-HQ-12: | | |
| | 0 μM | 3.5 μM | RA-HQ-12: | |
| | Zn: 0 μM | Zn: 128 μM | 3.5 μM | Zn: 128 μM |
| Erythromycin | 128 | 16 | 64 | 128 |
| Azithromycin | 128 | 32 | 64 | 128 |
| Tetracycline | 0.5 | 0.125 | 0.125 | 0.5 |
| Polymyxin B | 128 | 4^{#} | 32 | 64 |
| Colistin | >128 | 8 | 64 | >128 |
| Ampicillin | >128 | >128 | >128 | >128 |
| Vancomycin | 1 | 1 | 1 | 1 |
| Oxacillin* | >128 | 64 | >128 | >128 |
| | VRE | | | |
| | RA-HQ-12: | RA-HQ-12: | | |
| | 0 μM | 3.5 μM | RA-HQ-12: | |
| | Zn: 0 μM | Zn: 128 μM | 3.5 μM | Zn: 128 μM |
| Erythromycin | >128 | >128 | >128 | >128 |
| Azithromycin | >128 | >128 | >128 | >128 |
| Tetracycline | 128 | 128 | 128 | 128 |
| Polymyxin B | >128 | 4^{#} | 16 | >128 |
| Colistin | >128 | 2-4^{#} | 32 | >128 |
| Ampicillin | >128 | >128 | >128 | >128 |
| Vancomycin | >128 | >128 | >128 | >128 |
| Oxacillin* | >128 | >128 | >128 | >128 |
| | K. pneumoniae | | | |
| | RA-HQ-12: | | | |
| | 0 μM | RA-HQ-12: 16 μM | RA-HQ-12: | |
| | Zn: 0 μM | Zn: 64 μM | 16 μM | Zn: 64 μM |
| Erythromycin | >128 | >128 | >128 | >128 |
| Azithromycin | 32 | 32 | 32 | 32 |
| Tetracycline | 32 | 32 | 32 | 32 |
| Polymyxin B | 128 | 4^{#} | 128 | 128 |
| Colistin | 64 | 4^{#} | 32 | 64 |
| Ampicillin | >128 | >128 | >128 | >128 |
| Vancomycin | >128 | >128 | >128 | >128 |
| Oxacillin* | >128 | >128 | >128 | >128 |

*MIC for oxacillin against MRSA was determined in the presence of 2% NaCl as per CLSI guidelines.
Antibiotics concentrations where GAS, MRSA, VRE or K. pneumoniae MIC changes from resistant to sensitive in the presence of RA-HQ-12 + zinc are highlighted in bold^{#}.
MIC changes from resistant to intermediate sensitivity in the presence of RA-HQ-12 + zinc are highlighted in bold.

Efficacy of PBT2+Zinc(II) Ions in Combination with Antibiotics on Infection

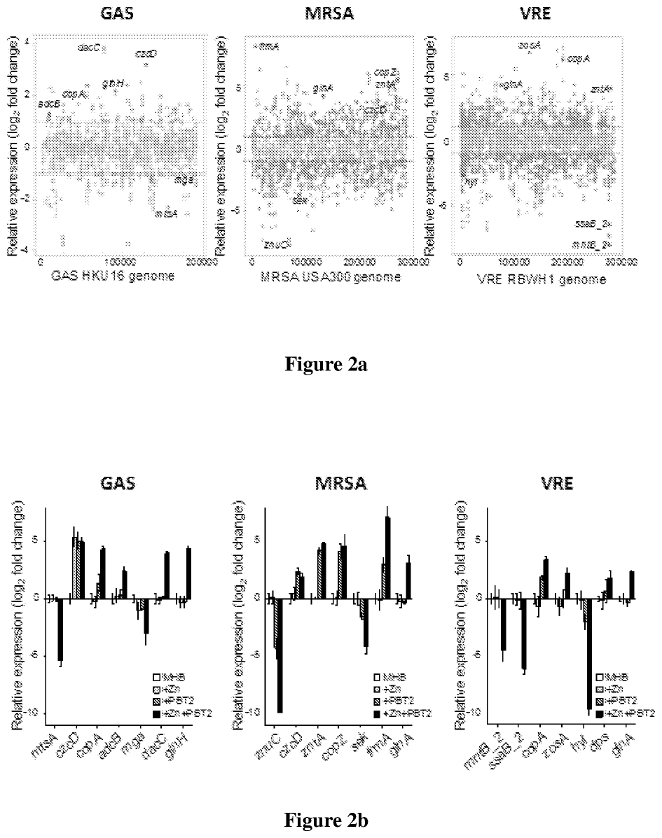

The efficacy of PBT2+zinc(II) ions in combination with antibiotics on infection, was investigated using the wound infection model. Neither antibiotic nor sub-inhibitory concentrations of PBT2+zinc(II) ions alone reduced bacterial burden at the site of infection. However, in combination, PBT2+zinc(II) ions resensitized GAS to tetracycline treatment and VRE to vancomycin treatment, thereby significantly reducing infection (FIG. 3).

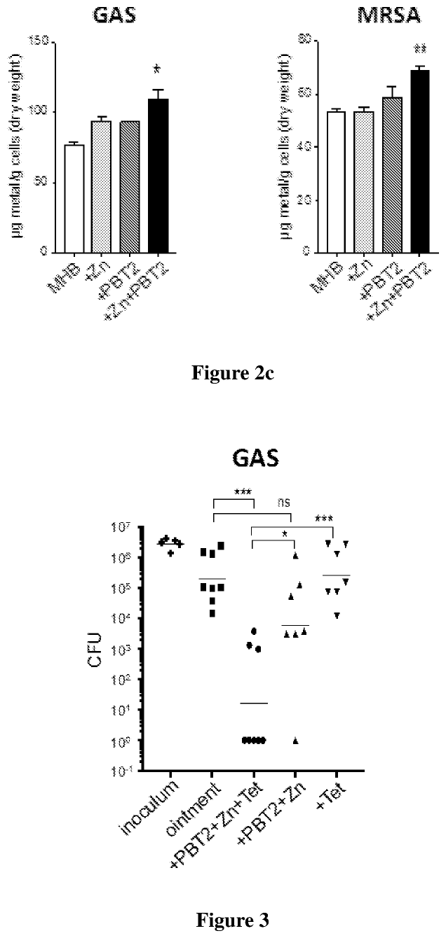

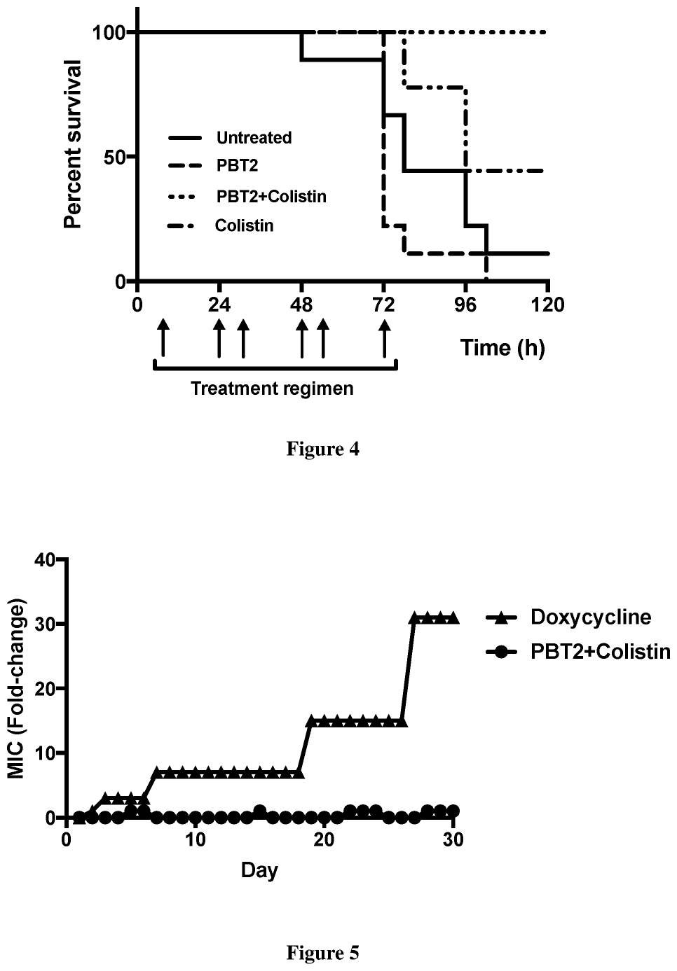

The efficacy of PBT2 in combination with colistin on a highly virulent colistin resistant Klebsiella pneumoniae strain 52.145ΔmgrB was investigated using the murine systemic (i.p.) infection model in CD1 mice. This demonstrates that PBT2+colistin treatment prevents colistin resistant K. pneumoniae strain 52.145ΔmgrB mortality in CD1 mice (FIG. 4). This observation paves the way for reduced colistin dosing in humans, potentially avoiding toxicity of this drug. There was no detection of K. pneumoniae MS6671 resistance against PBT2+colistin following serial passage for a period of 30 days in the presence of sub-inhibitory concentrations of each respective compound (FIG. 5).

PBT2, in combination with zinc(II) ions, possesses antibacterial activity and at sub-inhibitory concentrations reverses Gram-positive bacterial resistance against a number of important antibiotics. The mechanism of action underlying this effect appears to be changes in heavy metal homeostasis and profound disruption of essential virulence and metabolic systems that may weaken the capacity of these bacterial pathogens to cause infection.

The mechanism of action underlying this effect appears to be associated with changes in heavy metal homeostasis and profound disruption of essential virulence and metabolic systems, all of which may weaken the capacity of these bacterial pathogens to cause infection. Effects on antibiotic resistance were not universal. For instance, erythromycin resistance was reversed in MRSA but not in GAS. On the other hand, Gram-positive pathogens are intrinsically resistant to polymyxin and colistin (see, Li, J. et al. Colistin: the re-emerging antibiotic for multidrug-resistant Gram-negative bacterial infections. Lancet Infect Dis 6, 589-601, doi:10.1016/51473-3099(06)70580-1 (2006), Falagas, M. E. & Kasiakou, S. K. Colistin: the revival of polymyxins for the management of multidrug-resistant gram-negative bacterial infections. Clin Infect Dis 40, 1333-1341, doi:10.1086/429323 (2005)], yet PBT2+zinc treatment reversed resistance in GAS, MRSA and VRE. PBT2+zinc treatment reversed resistance in GAS, MRSA and VRE.

PBT2 is a safe-for-human-use zinc ionophore that has progressed to Phase 2 human clinical trials [see, e.g. Chakradhar, S. What's old is new: Reconfiguring known antibiotics to fight drug resistance. Nat Med 22, 1197-1199, doi:10.1038/nm1116-1197 (2016); Lannfelt, L. et al. Safety, efficacy, and biomarker findings of PBT2 in targeting Abeta as a modifying therapy for Alzheimer's disease: a phase IIa, double-blind, randomised, placebo-controlled trial. Lancet Neurol 7, 779-786, doi:10.1016/S1474-4422(08)70167-4 (2008)]. While zinc is used as a nutritional supplement and homeopathic remedy, at high concentrations zinc is known to be toxic. The delivery of zinc using the ionophore PBT2 can reduce the concentration of zinc required for efficacy to levels that may be tolerated physiologically [World Health Organization. Environmental Health Criteria 221: Zinc, <http://www.who.int/ipcs/publications/ehc/ehc_221/en/> (2001)]. Destabilisation of the bacterial physiology may circumvent antibiotic resistance by rescuing the function of antibiotics to which bacteria have become resistant.

Persons skilled in the art will appreciate that numerous variations and modifications will become apparent. All such variations and modifications which become apparent to persons skilled in the art, should be considered to fall within the spirit and scope that the invention broadly appearing before described.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1-29. (canceled)

30. A method of treating a bacterial infection in a subject comprising administering to a subject in need thereof an effective amount of a pharmaceutically acceptable zinc ionophore or a pharmaceutically acceptable derivative thereof, separately, concurrently or sequentially, and in any order, with administration of a therapeutically effective amount of an antibiotic or a pharmaceutically acceptable derivative thereof.

31. The method according to claim 30, wherein the zinc ionophore or a pharmaceutically acceptable derivative thereof, is in combination with an effective amount of a pharmaceutically acceptable zinc(II) salt or pharmaceutically acceptable solvate thereof, and wherein the antibiotic is not an aminoglycoside antibiotic.

32. The method according to claim 30, wherein the zinc ionophore is an 8-hydroxyquinoline derivative of Formula (I) as defined in WO2004/007461.

33. The method according to claim 30, wherein the zinc ionophore is a compound of Formula I: 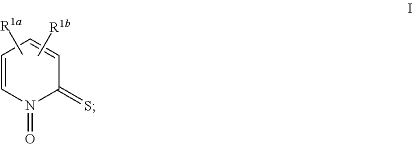
wherein R^{1a} and R^{1b} are independently H, halogen, OR^{2a}, SR^{2a}, CF₃, C₁₋₄alkyl, or NR^{2a}R^{2b}; R^{2a} and R^{2b} are independently H, or optionally substituted C₁₋₄alkyl;
or a pharmaceutically acceptable derivative thereof;
or a compound of Formula II: 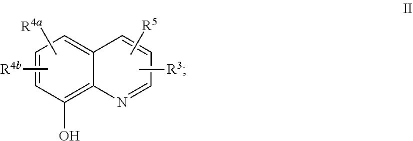
wherein:
R³ and R⁵ are independently H; optionally substituted C₁₋₆ alkyl; optionally substituted C₂₋₆ alkenyl; optionally substituted C₂₋₆ alkynyl; optionally substituted C₃₋₆ cycloalkyl; optionally substituted aryl; optionally substituted heterocyclyl; CN; OR⁶, SR⁶, COR⁶, CSR⁶, HCNOR⁶ or HCNNR⁶ in which R⁶ is H, optionally substituted C₁₋₆ alkyl, optionally substituted C₂₋₆alkenyl, optionally substituted C₂₋₆alkynyl, optionally substituted C₃₋₆cycloalkyl, optionally substituted aryl or optionally substituted heterocyclyl; NR⁸R⁹ or SO₂NR⁸R⁹ in which R⁸ and R⁹ are independently selected from H, optionally substituted C₁₋₆ alkyl, optionally substituted C₂₋₆alkenyl, optionally substituted C₂₋₆alkynyl, optionally substituted C₃₋₆cycloalkyl, optionally substituted aryl and optionally substituted heterocyclyl; CONR⁹R¹⁰ in which R⁹ is as defined above and R¹⁰ is optionally substituted C₁₋₆ alkyl, optionally substituted C₂₋₆alkenyl, optionally substituted C₂₋₆alkynyl, optionally substituted C₃₋₆cycloalkyl, optionally substituted aryl or optionally substituted heterocyclyl; CH₂CONR⁸R⁹ in which R⁸ and R⁹ are as defined above; and (CH₂)ₙNR⁹R¹¹ in which R⁹ is as defined above and R¹¹ is selected from optionally substituted C₁₋₆ alkyl, optionally substituted C₂₋₆alkenyl, optionally substituted alkynyl, optionally substituted C₃₋₆cycloalkyl, optionally substituted aryl, optionally substituted heterocyclyl and SO₂R¹² in which R¹² is optionally substituted C₁₋₆alkyl, optionally substituted C₂₋₆alkenyl, optionally substituted C₂₋₆alkynyl, optionally substituted C₃₋₆cycloalkyl, optionally substituted aryl or optionally substituted heterocyclyl, and n is 1 to 6;
R^{4a} and R^{4b} are independently H, optionally substituted C₁₋₄alkyl or halogen;
or a pharmaceutically acceptable derivative thereof.

34. The method according to claim 30, wherein the zinc ionophore is 5,7-dichloro-2-[(dimethylamino)methyl]-8-quinolinol (PBT2): 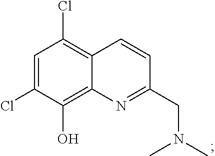 [SMILES: CN(C)Cc1ccc2c(Cl)cc(Cl)c(O)c2n1]
or a pharmaceutically acceptable derivative thereof.

35. The method according to claim 30, wherein the antibiotic is a carbapenem, a cephalosporin, a glycopeptide, a lincosamide, a macrolide, a monobactam, a nitrofuran, an oxazolidinone, a penicillin, a polypeptide, a quinolone, a sulphonamide, or a tetracycline; or chloramphenicol, fosfomycin, fusidic acid, metronidazole, mupirocin, thiamphenicol, tigecycline, tinidazole, or trimethoprim, or a pharmaceutically acceptable derivative of any one thereof.

36. The method according to claim 30, wherein the antibiotic is a polypeptide antibiotic or a pharmaceutically acceptable salt thereof.

37. The method according to claim 36, wherein the polypeptide antibiotic is a polymyxin.

38. The method according to claim 30, wherein the antibiotic is colistin, polymyxin B, tetracycline, tigecycline, doxycycline, oxacillin, erythromycin, ampicillin, vancomycin, penicillin, or chloramphenicol, or a pharmaceutically acceptable derivative of any one thereof.

39. The method according to claim 30, wherein the bacterial infection involves a Gram positive bacterium.

40. The method according to claim 30, wherein the bacterial infection involves a Gram negative bacterium.

41. The method according to claim 30, wherein a pathogenic bacterium involved in the bacterial infection is a Klebsiella spp, an Erythromycin-resistant Group A Streptococcus spp., methicillin-resistant Staphylococcus aureus (MRSA), vancomycin-resistant Enterococcus (VRE), Escherichia coli, Streptococcus pneumonia, Neisseria spp., Acinetobacter spp., and Pseudomonas spp.

42. A method of restoring the sensitivity of a resistant pathogenic bacterium to an antibiotic or for inhibiting resistance of a pathogenic bacterium to an antibiotic comprising the steps of:
administering an effective amount of a pharmaceutically acceptable zinc ionophore or a pharmaceutically acceptable salt thereof;
administering an effective amount of an antibiotic or a pharmaceutically acceptable salt thereof; and, optionally,
administering an effective amount of a pharmaceutically acceptable zinc(II) salt or a pharmaceutically acceptable solvate thereof;
to a subject in need thereof;
wherein the steps are concurrent, sequential or separate and may performed in any order.

43. A pharmaceutical composition comprising a pharmaceutically acceptable zinc ionophore or a pharmaceutically acceptable derivative thereof and an antibiotic or a pharmaceutically acceptable derivative thereof; and, optionally, a pharmaceutically acceptable zinc(II) salt or pharmaceutically acceptable solvate thereof; and
a pharmaceutically acceptable carrier.

44. A method of treating a bacterial infection in a subject comprising the administration of a therapeutically effective and non-toxic amount of a pharmaceutical composition according to claim 43.

45. The composition according to claim 43, wherein the zinc ionophore is a compound of Formula I: 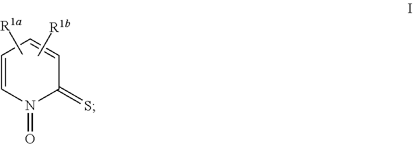
wherein R^{1a} and R^{1b} are independently H, halogen, OR^{2a}, SR^{2a}, CF₃, C₁₋₄alkyl, or NR^{2a}R^{2b}; R^{2a} and R^{2b} are independently H, or optionally substituted C₁₋₄alkyl;
or a pharmaceutically acceptable derivative thereof;
or a compound of Formula II: 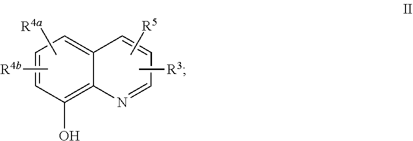
wherein:
R³ and R⁵ are independently H; optionally substituted C₁₋₆ alkyl; optionally substituted C₂₋₆ alkenyl; optionally substituted C₂₋₆alkynyl; optionally substituted C₃₋₆cycloalkyl; optionally substituted aryl; optionally substituted heterocyclyl; CN; OR⁶, SR⁶, COR⁶, CSR⁶, HCNOR⁶ or HCNNR⁶ in which R⁶ is H, optionally substituted C₁₋₆ alkyl, optionally substituted C₂₋₆alkenyl, optionally substituted C₂₋₆alkynyl, optionally substituted C₃₋₆cycloalkyl, optionally substituted aryl or optionally substituted heterocyclyl; NR⁸R⁹ or SO₂NR⁸R⁹ in which R⁸ and R⁹ are independently selected from H, optionally substituted C₁₋₆ alkyl, optionally substituted C₂₋₆alkenyl, optionally substituted C₂₋₆alkynyl, optionally substituted C₃₋₆cycloalkyl, optionally substituted aryl and optionally substituted heterocyclyl; CONR⁹R¹⁰ in which R⁹ is as defined above and R¹⁰ is optionally substituted C₁₋₆ alkyl, optionally substituted C₂₋₆alkenyl, optionally substituted C₂₋₆alkynyl, optionally substituted C₃₋₆cycloalkyl, optionally substituted aryl or optionally substituted heterocyclyl; CH₂CONR⁸R⁹ in which R⁸ and R⁹ are as defined above; and (CH₂)ₙNR⁹R¹¹ in which R⁹ is as defined above and R¹¹ is selected from optionally substituted C₁₋₆ alkyl, optionally substituted C₂₋₆alkenyl, optionally substituted alkynyl, optionally substituted C₃₋₆cycloalkyl, optionally substituted aryl, optionally substituted heterocyclyl and SO₂R¹² in which R¹² is optionally substituted C₁₋₆alkyl, optionally substituted C₂₋₆alkenyl, optionally substituted C₂₋₆alkynyl, optionally substituted C₃₋₆ cycloalkyl, optionally substituted aryl or optionally substituted heterocyclyl, and n is 1 to 6;
R^{4a} and R^{4b} are independently H, optionally substituted C₁₋₄alkyl or halogen;
or a pharmaceutically acceptable derivative thereof.

46. The composition according to claim 43, wherein the zinc ionophore is
5,7-dichloro-2-[(dimethylamino)methyl]-8-quinolinol (PBT2): 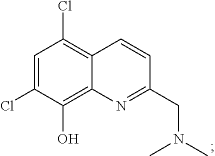 [SMILES: CN(C)Cc1ccc2c(Cl)cc(Cl)c(O)c2n1]
or a pharmaceutically acceptable derivative of either thereof.

47. The composition according to claim 43, wherein the antibiotic is a carbapenem, a cephalosporin, a glycopeptide, a lincosamide, a macrolide, a monobactam, a nitrofuran, an oxazolidinone, a penicillin, a polypeptide, a quinolone, a sulphonamide, or a tetracycline; or chloramphenicol, fosfomycin, fusidic acid, metronidazole, mupirocin, thiamphenicol, tigecycline, tinidazole, or trimethoprim, or a pharmaceutically acceptable derivative of any one thereof.

48. The composition according to claim 43 wherein the antibiotic is colistin, polymyxin B, tetracycline, tigecycline, doxycycline, oxacillin, erythromycin, ampicillin, vancomycin, penicillin, or chloramphenicol, or a pharmaceutically acceptable derivative of any one thereof.

49. The composition according to claim 43 wherein the antibiotic is a polypeptide antibiotic or a pharmaceutically acceptable derivative thereof.
